(19) United States
(12) Patent Application Publication
(10) Pub. No.: US 2017/0209605 A1
(43) Pub. Date: Jul. 27, 2017

(54) CARBOHYDRATE FUNCTIONALIZED CATANIONIC SURFACTANT VESICLES FOR DRUG DELIVERY

(71) Applicant: University of Maryland, College Park, College Park, MD (US)

(72) Inventors: Douglas S English, College Park, MD (US); Philip R DeShong, College Park, MD (US); Daniel C Stein, College Park, MD (US); Sara Lioi, College Park, MD (US); Ju-Hee Park, College Park, MD (US); Emily J Danoff, College Park, MD (US); Glen B Thomas, College Park, MD (US)

(73) Assignee: University of Maryland, College Park, College Park, MD (US)

(21) Appl. No.: 15/480,116

(22) Filed: Apr. 5, 2017

Related U.S. Application Data
(63) Continuation of application No. 12/673,844, filed on Mar. 15, 2011, now abandoned.
(60) Provisional application No. 60/956,406, filed on Aug. 17, 2007.
(60) Provisional application No. 60/987,227, filed on Nov. 12, 2007.
(60) Provisional application No. 61/080,561, filed on Jul. 14, 2008.

(51) Int. Cl. A61K 51/12; A61K 49/00; A61K 51/08; A61K 51/04; A61K 39/00; A61K 39/39; A61K 39/02; A61K 31/704; G01N 33/68; G01N 33/58; G01N 33/80; B01J 13/10; B01J 13/20; A61J 1/14; A61J 1/03; A61K 48/00

(52) U.S. Cl. CPC A61K 51/1234; A61K 48/0008; A61K 49/0084; A61K 51/088; A61K 51/0491; A61K 51/0474; A61K 49/0054; A61K 39/00; A61K 39/39; A61K 39/02; A61K 31/704; A61K 49/0021; A61K 49/0041; A61K 47/48815; A61K 47/4803; A61K 47/48215; A61K 47/4823; A61K 47/4833; G01N 33/68; G01N 33/586; G01N 33/80; B01J 13/10; B01J 13/203; A61J 1/14; A61J 1/03; A61K 2039/55583; A61K 2039/55516; A61K 2039/575; A61K 2039/622; G01N 2333/4724

(57) ABSTRACT

Carbohydrate functionalized catanionic vesicles that include a glycoconjugate and/or peptidoconjugate for vaccination or drug delivery, methods for forming these, and methods of using these.

### CROSS-REFERENCE TO RELATED APPLICATIONS

This is a continuation of U.S. application Ser. No. 12/673,844, filed Mar. 15, 2011, which is a National Stage entry of International Application No. PCT/US2008/009824, filed Aug. 18, 2008, which claims the benefit of U.S. Provisional Application Nos. 60/956,406, filed Aug. 17, 2007, 60/987,227, filed Nov. 12, 2007, and 61/080,561, filed Jul. 14, 2008, each of which is incorporated herein in its entirety.

This invention was made with Government support under CTS0608906 awarded by the National Science Foundation. The United States Government has certain rights in this invention.

### BACKGROUND OF THE INVENTION

Liposomal encapsulation of a drug can improve drug solubility and increase circulation time by altering the biodistribution of the drug. Targeting of liposomes in vivo can be achieved by modifying the bilayer surface with antibodies or ligands, thereby directing the drug toward a specific tissue type (Allen, T. M.; Moase, E. H. Advanced Drug Delivery Reviews 1996, 21, 117). Targeted delivery of toxic drugs, such as chemotherapeutic agents, can decrease the amount of drug that accumulates in sensitive tissues and organs, and thereby reduce the toxic effects of the drug resulting in an improvement in therapeutic index. Liposomal preparations approved for clinical use include Doxil and DepoCyt for cancer chemotherapeutic drugs, DepoDur for morphine delivery and Ambisome, which is a formulation for liposomal delivery of antifungal agents.

However, liposomes formed by sonication or extrusion are essentially kinetically-trapped, nonequilibrium structures, that tend to fuse or rupture to form lamellar phases. In the fusion process, the contents of the phospholipid vesicles are released.

### SUMMARY

In an embodiment according to the invention, a catanionic surfactant vesicle includes a bilayer comprising a cationic surfactant, an anionic surfactant, and a bioconjugate. A bioconjugate can be, for example, a glycoconjugate, a peptidoconjugate, or a conjugate with both glyco and peptide groups. The bilayer can have a net surface charge. The bilayer can have an inner surface and an outer surface. The bioconjugate can include a carbohydrate and/or peptide moiety and a hydrophobic group. At least a portion of the hydrophobic group can be within the bilayer. The carbohydrate and/or peptide moiety can be on the outer surface of the bilayer. The bioconjugate can be, for example, a lipid oligosaccharide or a lipid polysaccharide. The hydrophobic group of the bioconjugate can include an alkyl chain. The catanionic surfactant vesicle can include an inner pool bounded by the inner surface of the bilayer.

The catanionic surfactant vesicle can include a solute molecule or a solute ion having a charge. The solute molecule or solute ion can be within the inner pool and/or the bilayer. The net surface charge of the bilayer can be opposite to that of the solute ion. The solute molecule or solute ion can be, for example, a dye, a radionuclide, a pharmaceutical agent, a biotherapeutic agent, a chemotherapeutic agent, a radiotherapeutic agent, and combinations a metal, a natural product, a peptide, an oligopeptide, a polypeptide, a saccharide, an oligosaccharide, a polysaccharide, a nucleotide, an oligonucleotide, a polynucleotide, DNA, RNA, carboxyfluoroscein (CF), sulfarhodamine 101 (SR 101), Lucifer yellow (LY), rhodamine 6G (R6G), Doxorubicin (Dox), derivatives of these, or combinations.

The carbohydrate and/or peptide moiety of the bioconjugate can be bound to the receptor on the surface of a cell. For example, a carbohydrate moiety of the bioconjugate can be bound to a lectin.

In an embodiment according to the invention, a catanionic vesicle library can include at least two catanionic surfactant vesicles. Each catanionic surfactant vesicle can include an independently selected bioconjugate. A first catanionic surfactant vesicle including a first bioconjugate can include a solute molecule or solute ion that is different than a solute molecule or solute ion included in a second catanionic surfactant vesicle including a second bioconjugate different than the first bioconjugate.

In an embodiment according to the invention, a blood-typing system can include a first catanionic surfactant vesicle that includes a first dye. A glycoconjugate of the first catanionic surfactant vesicle can bind to a first blood-type antibody specific to a first blood-type antigen. The blood-typing system can include a second catanionic surfactant vesicle that includes a second dye. The glycoconjugate of the second catanionic surfactant vesicle can bind to a second blood-type antibody specific to a second blood-type antigen. For example, the first blood type antibody can be anti-A, and the second blood type antibody can be anti-B.

In an embodiment according to the invention, a lectin detection system can include a catanionic surfactant vesicle that includes a dye. A glycoconjugate of the catanionic surfactant vesicle can be selected to bind to a lectin sought to be detected, for example, a predetermined lectin.

In an embodiment according to the invention, a vaccine can include a physiologically acceptable carrier and a catanionic surfactant vesicle that includes a bioconjugate.

In an embodiment according to the invention, a kit can include a premeasured amount of an anionic surfactant in a first labeled container, a premeasured amount of a cationic surfactant in a second labeled container, and a premeasured amount of a bioconjugate in a third labeled container. The premeasured amounts of the anionic surfactant, cationic surfactant, and bioconjugate can be selected, so that when the anionic surfactant, cationic surfactant, and bioconjugate are added to a predetermined amount of water, catanionic surfactant vesicles are formed.

A method of making a bioconjugate-decorated catanionic vesicle according to the invention can include combining an anionic surfactant, a cationic surfactant, and a bioconjugate with water to form a bioconjugate-decorated catanionic vesicle. The bioconjugate-decorated catanionic vesicle can have a bilayer with an inner surface and an outer surface. The inner surface of the bilayer can bound an inner pool. The bioconjugate-decorated catanionic vesicle can include the anionic surfactant and the cationic surfactant. At least a portion of the hydrophobic group can be within the bilayer, and the carbohydrate moiety can be on the outer surface of the bilayer. The charge of a solute ion can be determined. The proportion of the anionic surfactant to the cationic surfactant can be selected so that the bilayer of the catanionic vesicle has a net surface charge opposite to that of the solute ion. The solute ion can be combined with the anionic surfactant, cationic surfactant, and bioconjugate at the same time to produce a bioconjugate-decorated catanionic vesicle with the solute ion within the inner pool and/or the bilayer. Alternatively, the solute ion can be combined with an already formed bioconjugate-decorated catanionic vesicle to sequester the solute ion within the inner pool and/or the bilayer.

A method of introducing an agent into a cell according to the invention includes contacting the cell with a composition comprising catanionic surfactant vesicles bearing bioconjugates and having the agent sequestered in the bilayer and/or the inner pool. The cell can include a lectin, a carbohydrate-binding, and/or a peptide binding site that binds the bioconjugate. The agent can be, for example, a dye, a radionuclide, a pharmaceutical agent, a biotherapeutic agent, a chemotherapeutic agent, a radiotherapeutic agent, a metal, a natural product, a peptide, an oligopeptide, a polypeptide, a saccharide, an oligosaccharide, a polysaccharide, a nucleotide, an oligonucleotide, a polynucleotide, DNA, RNA, a derivatives of these, or a combination of these. In a method of gene therapy according to the invention, the agent can be a nucleic acid.

A method for eliciting or stimulating an immune response in a subject according to the invention includes administering to the subject an amount of a bioconjugate-decorated catanionic surfactant vesicle in a physiologically acceptable carrier effective to elicit or stimulate the immune response. The carbohydrate and/or peptide group of the bioconjugate can bind to an immune receptor to elicit or stimulate the immune response. The immune response elicited or stimulated can be an immunoprotective response.

A method for determining the separation distance of carbohydrate binding sites on a sample lectin according to the invention can include the following. A set of catanionic surfactant vesicles conjugated with a glycoconjugate comprising a carbohydrate moiety that is a ligand for the sample lectin can be produced. Within the set, catanionic surfactant vesicles can be formed over a range of glycoconjugate mole fractions. The initial rate of reaction between each catanionic surfactant vesicle functionalized with the glycoconjugate in the set and the sample lectin can be determined with a turbidity assay. The value of carbohydrate binding site separation in a collision model can be determined that provides the best fit to the initial rate of reaction as a function of the mole fraction of glycoconjugate data. This value of carbohydrate binding site separation in the collision model can be taken as representative of the separation distance of carbohydrate binding sites on the sample lectin. An analogous method can be applied to determine the separation distance of peptide binding sites on a biological molecule or structure.

A method of detecting receptors on a sample according to the invention can include the following. Catanionic surfactant vesicles can be administered to the sample. Excess catanionic surfactant vesicles can be flushed from the sample. A characteristic signal of a label of the catanionic surfactant vesicles can be imaged. For example, such a characteristic signal can be light signal (of a label that is a dye or a fluorescent dye) or nuclear radiation (of a label that is a radionuclide). Regions of the sample that display the characteristic signal of the label can be associated with binding of the catanionic surfactant vesicles to the sample and, therefore, the presence of the receptors on the sample. The catanionic surfactant vesicles can include a bilayer having an inner surface and an outer surface comprising a cationic surfactant, an anionic surfactant, and a bioconjugate. The bioconjugate can include a carbohydrate and/or peptide moiety and a hydrophobic group. At least a portion of the hydrophobic group can reside within the bilayer and the carbohydrate moiety can be present on the outer surface. The inner surface can bound an inner pool and the label can be sequestered in the bilayer and/or the inner pool. The carbohydrate moiety can be capable of binding with the receptor of the sample.

For example, a method of detecting cancer cells in a can include the following. Catanionic surfactant vesicles in a physiologically acceptable carrier can be administered to the subject. The catanionic surfactant vesicles can be allowed to bind with receptors on the cancer cells. Unbound catanionic surfactant vesicles can be allowed to be excreted from the subject. A characteristic signal of a label of the catanionic surfactant vesicles can be imaged. Regions of the subject displaying the characteristic signal of the label can be associated with binding of the catanionic surfactant vesicles and, therefore, the presence of cancer cells. The catanionic surfactant vesicles can include a bilayer having an inner surface and an outer surface that includes a cationic surfactant, an anionic surfactant, and a bioconjugate. The bioconjugate can include a carbohydrate and/or peptide moiety and a hydrophobic group. At least a portion of the hydrophobic group can reside within the bilayer and the carbohydrate moiety can be present on the outer surface. The inner surface can bounds an inner pool. The label can be sequestered in the bilayer and/or the inner pool. The carbohydrate moiety can be capable of binding with the receptors on the cancer cells.

A method of treating cancer in a subject according to the invention can include the following. Catanionic surfactant vesicles in a physiologically acceptable carrier can be administered to a subject. The catanionic surfactant vesicles can be allowed to bind with receptors on the cancer cells. A chemotherapeutic, radiotherapeutic, and/or biotherapeutic agent can be sequestered in the bilayer and/or the inner pool of the catanionic surfactant vesicles. The carbohydrate moiety can be capable of binding with the receptors on the cancer cells.

A method of treating a microbial infection in a subject according to the invention can include the following. Catanionic surfactant vesicles in a physiologically acceptable carrier can be administered to a subject. The catanionic surfactant vesicles can be allowed to bind with receptors on microbes of the microbial infection. A pharmaceutical agent can be sequestered in the bilayer and/or the inner pool of the catanionic surfactant vesicles. The carbohydrate moiety can be capable of binding with the receptors on the microbes.

### BRIEF DESCRIPTION OF THE DRAWINGS

FIG. 1. Left: Cut-away view of a surfactant based vesicle formed from a two-component mixture of single-tailed surfactants. Right: A surfactant vesicle that includes additional components including a nonionic carbohydrate based surfactant, e.g., a bioconjugate, used to decorate the vesicle exterior for targeting purposes.

FIG. 2 presents the chemical structures of several N-linked glycoconjugates for the surface functionalization of catanionic vesicles.

FIG. 3 presents graphs of absorbance data from size exclusion chromatography (SEC) and of intensity data from dynamic light scattering (DLS) measurements. (A) Sodium dodecylbenzenesulfonate (SDBS)-rich vesicles with C₈-glucose; (B) SDBS-rich vesicles with C₈-lactose.

FIG. 4 presents results from SEC analysis of sodium dodecylbenzenesulfonate (SDBS)-rich vesicles with C₁₂-glucose; A) Measured values of scattering intensity (red circles) and UV-vis intensity for colorimetric detection of glycoconjugates (blue triangles) as a function of eluted fraction. B) Plot of detected glucose (proportional to UV-vis signal of colorimetric assay) versus initial mole fraction of C₁₂-Glu.

FIG. 5 presents a graph comparing the release of solutes sequestered in liposomes and catanionic vesicles as a function of time, R(t).

FIG. 6 presents evidence of negatively-charged vesicles being used to segregate two mixed ionic dyes. The dye mixture was combined with a negatively-charged vesicles which sequestered the oppositely charged dye, Rhodamine 6G. The mixture of dyes and vesicles were separated using size exclusion chromatography. The yellow dye is the anionic dye carboxyflouresceine which elutes behind the band containing the vesicle-bound cationic dye rhodamine 6G which has appears pink.

FIG. 7 presents fluorescence correlation spectroscopy (FCS) results from studies of electrostatic adsorption on vesicle bilayers. (A) represents data acquired with single-photon time-tagging methods. The decay times increase with increasing vesicle concentration as more fluorescent probe molecules adsorb to the vesicle surface. The fits to these decays provide a quantitative measurement of the distribution of free and bound dyes. B) Binding isotherms are constructed from the FCS decay curves. These isotherms provide quantitative information on how electrostatic binding varies with parameters such as charge ratio, counter-ion identity and ionic strength.

FIG. 8 presents the results from lectin-induced agglutination studies with carbohydrate functionalized surfactant vesicles. (A) represents titration results using Con A, (B) represents titration results using PNA. In both graphs, the circle represents C₈-glucose modified vesicles, the square represents C₈-lactose modified vesicles, and the cross represents bare vesicles.

FIG. 9 Change in turbidity of vesicles as a function of added Con A concentration. Absorbance values of about 1.2 indicated the approximate saturation point of aggregation. As the mole fraction of C₁₂-Glucose is lowered more Con A is required to induce aggregation. This illustrates control over surface coverage and emphasizes the need for high ligand density to induce multivalent binding by ConA.

FIG. 10 presents the effect of carbohydrate length on Con A-induced agglutination. (A) Final turbidity as a function of Con A concentration. (B) Turbidity as a function of time with [Con A]=5.0 M.

FIG. 11 presents binding rates of Con A as a function of carbohydrate surface coverage used to elucidate the multivalent binding of lectins.

FIG. 12 presents an illustration of how lectins cause carbohydrate functionalized vesicles to agglutinate. The top panel shows a cartoon depicting the cross-linking of vesicles by a multivalent ligand such as Con A. The lower panels shows cryoscopic transmission electron microscopy images of vesicles with 0.005 mole fraction C₁₂-Glucose. Before Con A is added the vesicles are unilamellar and spherical. After Con A is added the vesicles are aggregated.

FIG. 13 presents a graphical representation of the synthetic route for the preparation of glycoconjugates.

FIG. 14 presents images of catanionic surfactant vesicles in the presence of Neisseria gonorrhoeae cells.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without parting from the spirit and scope of the invention. All references cited herein are incorporated by reference as if each had been individually incorporated.

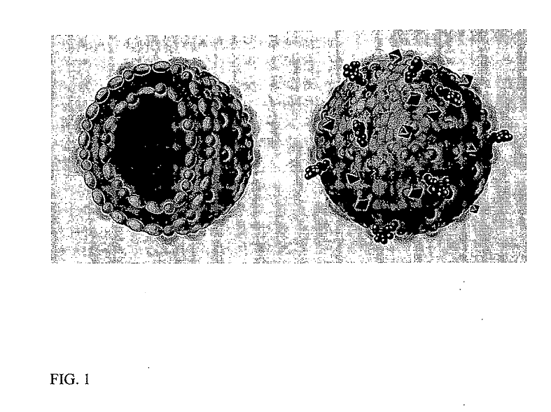

Embodiments according to the present invention include the use of surfactant vesicles with thermodynamic, cell-targeting, and functionalization properties that indicate their use in research, diagnostic, and therapeutic applications. The word “liposome” is used to refer to conventional vesicles in which the major components are phospholipids. The word “vesicle” or “catanionic vesicle” is used to refer to spontaneously formed unilamellar bilayers enclosing an inner water pool in which the primary major components are two oppositely charged single-tailed surfactants. FIG. 1 presents a cartoon of the surfactant vesicle system used in this patent.

Embodiments according to the invention include carbohydrate and/or peptide functionalized surfactant vesicles formed from mixtures of oppositely-charged single-tailed surfactants (commonly referred to as “catanionic” vesicles) and bioconjugates, for example, glycoconjugates, such as alkylated carbohydrates. For example, these vesicles can sequester and separate charged biomolecules in solution. To add increased biofunctionality to these vesicles, or to target the delivery of sequestered molecules, these catanionic vesicles can be enhanced with the addition of one or more bioconjugates, both charged and non-ionic, in order to interact with natural or artificial carbohydrate and/or protein recognition systems. These carbohydrate- and/or protein-functionalized vesicles present binding residues to an actual cell surface and facilitate multivalent interactions. The recognition process for a carbohydrate is fundamentally different than protein-protein or antibody-antigen interactions at cell surfaces in that carbohydrate recognition is a multivalent process. Since each binding event of a carbohydrate-mediated system involves weak interactions (H-bonding), then the receptors involved must establish multiple interactions to achieve high selectivity (Mammen, S. K. Choi and G. M. Whitesides, Angew. Chem. Int. Ed., 1998, 37, 2755-2794). Accordingly, the recognition of glycosyl residues on the cell surface requires clustering or a high density of surface receptors. It is this multivalent binding process of oligosaccharide-mediated recognition that can in certain cases be advantageous in comparison with recognition strategies associated with other biomolecules such as proteins or nucleic acids.

Surfactant vesicles for surface presentation, encapsulation, and delivery purposes can have several advantages over conventional phospholipids including lower cost, ease of preparation, and inherent stability. “Catanionic” surfactant vesicles can be spontaneously generated when a mixture of cationic and anionic surfactants are combined with water under appropriate proportions. Vesicle formation under such conditions can be spontaneous and fairly rapid (<12 h) and yield vesicles that are thermodynamically stable. These surfactant vesicles can be stable for long periods. By contrast, phospholipid liposomes formed by sonication or extrusion are essentially kinetically-trapped, nonequilibrium structures, that tend to fuse or rupture to form lamellar phases, in the process, releasing their contents.

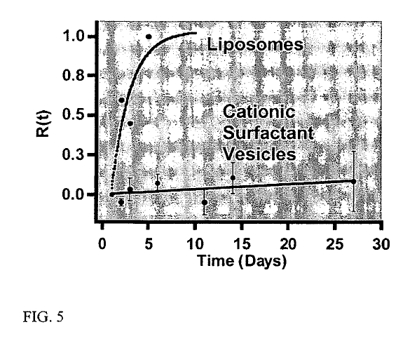

Spontaneously forming catanionic vesicles formed from the anionic surfactant sodium dodecylbenzenesulfonate (SDBS) and the cationic surfactant cetyltrimethylammonium tosylate (CTAT) capture charged organic solutes with extremely high efficiency and with very slow spontaneous release rates (FIG. 5). The strong electrostatic interactions between catanionic vesicles and ionic solutes may be used, for example, to separate an oppositely charged solute from a solute mixture. To demonstrate this, vesicles were prepared with equimolar mixtures of two solutes, one cationic (R6G) and the other anionic (CF). The total solute concentration was maintained at either 0.5 or 1.0 mM, and the experiments were done with both positively-charged vesicles (V⁺) and negatively-charged vesicles (V⁻) vesicles. Experiments with these solute mixtures were performed and analyzed using size exclusion chromatography to determine the amount and type of dye captured by the vesicle.

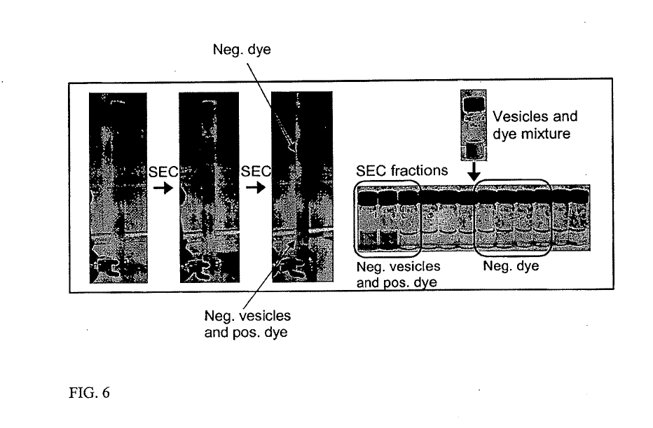

Results from an equimolar mixture of CF and R6G, at a total dye concentration of 0.5 mM, in V⁻ vesicles are shown in FIG. 6. In this case, the V⁻ vesicle band emerging out of the SEC column contained 88% of the R6G, while the amount of CF in this band was negligible. Thus, the V⁻ vesicles were able to bind and separate the cationic dye from the dye mixture. Thus, surfactant vesicles can be used to separate ionic compounds. Similar experiments with a total dye concentration of 1.0 mM CF and R6G were conducted, and similar results were obtained. Separation experiments were conducted using an anionic dye, LY, and a cationic drug, Dox, and very efficient separation was observed using catanionic vesicles, much as illustrated in FIG. 6. When V⁺ vesicles were used in place of V⁻ vesicles 31% of the anionic CF was carried through the SEC column within the V⁺ vesicle band, and no detectable R6G emerged with the vesicles. In short, the V⁺ vesicles were able to selectively capture the anionic dye and separate it from the dye mixture. The high carrying capacity of SDBS/CTAT vesicles is understood to be due to strong electrostatic interactions between the charged vesicle bilayer and the organic solute. Vesicles can be formed with either an excess of cationic or anionic surfactant and used to carry charged solutes. Preparations formed from surfactant vesicles have a much longer shelf life relative to liposomal preparations due to the superior stability of surfactant vesicles (FIG. 5). The catanionic surfactant vesicles studied were approximately 140 nm in diameter. These catanionic surfactant vesicles are candidates for delivering molecular payloads to cells, for example, for fluorescent staining or drug delivery.

Thus, drug and dye molecules are held in catanionic surfactant vesicles with high efficiency. A mechanism for sequestration is understood to be based on electrostatic interactions between the solute and the vesicle bilayer. Cationic vesicles efficiently sequester anionic solutes whereas anionic vesicles efficiently sequester cationic solutes. For instance, SDBS-rich vesicles capture and hold the positively charged dye rhodamine 6G or the positively charged drug doxorubicin. The release of the sequestered molecules can occur in two phases: an initial burst release occurring over a few days can be followed by a slow release occurring over months. These release characteristics make SDBS-rich vesicles, and surfactant vesicles in general, strong candidates for drug delivery or other biotechnological applications requiring the controlled release of molecular payloads, compared to traditional liposomal carriers. FIG. 5 shows the release profile for carboxyfluorescein (CF) from positively-charged vesicles, compared with the release of the same molecules from conventional phospholipid molecules. Negatively-charged surfactant vesicles such as those prepared with excess SDBS are well-suited for use as diagnostic agents, because strategies for inducing specific interactions with cell surfaces can be engineered.

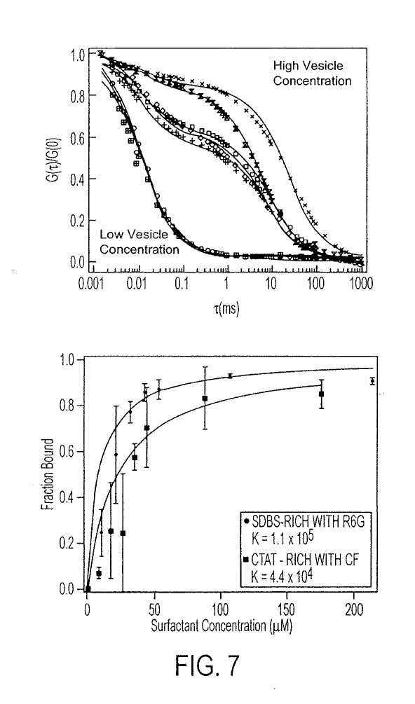

Quantitative experiment to determine solute binding to the charged exteriors of surfactant vesicles fluorescence correlation spectroscopy (FCS) for evaluating the fraction of solute molecules that are strongly bound to the vesicle surface have been conducted. The diffusion time of fluorescent cargo molecules as they pass through a tightly-focused laser beam was measured. The diffusion time is short (˜100 μs) for an unbound cargo molecule and much longer (˜100 ms) for a cargo molecule that is strongly bound to a surfactant vesicle. After determining the fraction of molecules that are bound as a function of vesicle concentration, a binding isotherm was constructed. The experiments were conducted by obtaining autocorrelation curves (G(τ)) from fluorescence fluctuations. The decay time of the autocorrelation curve increases as more dye is bound to the vesicle exterior. FIG. 7 shows examples of results from this method. FIG. 7 shows autocorrelation decay data for different concentrations. Each decay curve is fit to an equation which describes the diffusion of two species: 1) free dye and 2) vesicle bound dye. The best fit to this equation yields the fraction of dye which is bound to the vesicle,

$$G ( τ ) = f \cdot ( \frac{1}{1 + \frac{τ}{{τ}_{v}}} ) \cdot {( \frac{1}{1 + {ω}^{2} \frac{τ}{{τ}_{v}}} )}^{\frac{1}{2}} + ( 1 - f ) \cdot ( \frac{1}{1 + \frac{τ}{{τ}_{p}}} ) \cdot {( \frac{1}{1 + {ω}^{2} \frac{τ}{{τ}_{p}}} )}^{\frac{1}{2}}$$

After determining the fraction of molecules that are bound as a function of vesicle concentration, a binding isotherm was constructed, FIG. 7B. This analysis allows the quantification of binding constant, K, for various dye-vesicle mixtures.

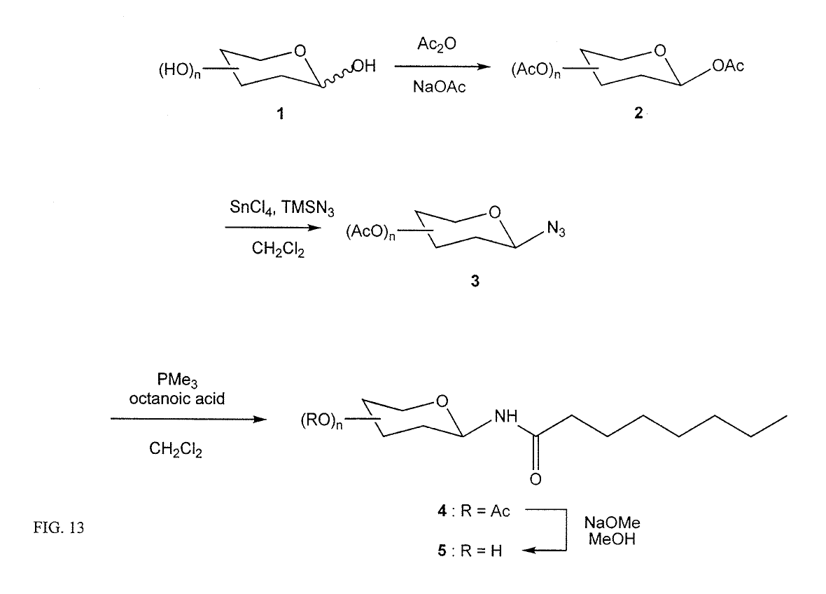

An embodiment according to the present invention includes methods for engineering specific interactions through the incorporation of bioconjugate molecules into catanionic vesicles. Glycoconjugates were synthesized using the approach illustrated in FIG. 13. Functionalization with carbohydrates takes advantage of the many cell surface receptors that have evolved to selectively identify carbohydrates and can be exploited for targeted delivery. The robust nature of catanionic surfactant vesicles allows their surfaces to be easily modified by simple hydrophobic insertion. For example, studies have shown the use of hydrophobically modified chitosan to form a crosslinked vesicle/polymer gel. Studies were carried out using both C₈-glycoconjugate and C₁₂-glycoconjugate for incorporation into the vesicle bilayer of catanionic surfactant vesicles. The accessibility of the carbohydrates to receptors in solution using well-established lectin binding assays was evaluated.

A glycoconjugate can include a carbohydrate that is covalently linked to another chemical species. Examples of glycoconjugates include glycoproteins, glycopeptides, peptidoglycans, glycolipids, lipopolysaccharides, and carbohydrates covalently linked to one or more alkyl chains.

A carbohydrate or saccharide can include monosaccharides, oligosaccharides, and polysaccharides. An oligosaccharide can be formed of a few covalently linked, and a polysaccharide can be formed of many covalently linked monosaccharide units. A monosaccharide can be formed of an aldehyde or ketone with attached hydroxyl groups. Examples of monosaccharides include aldohexoses, such as glucose, aldopentoses, such as ribose, and ketohexoses, such as fructose. Monosaccharides can exist in a straight-chain or in a cyclic form, e.g., a furanose or pyranose. Carbohydrates can be displayed on the outer surface of the membranes of cells. For example, carbohydrates displayed in antigens on the surface of erythrocytes or red blood cells are responsible for the blood type of an animal.

The carbohydrate and/or peptide moiety of a bioconjugate can be selected to bind with a receptor on a target cell or another target structure. For example, the carbohydrate moiety can be selected to bind with a carbohydrate receptor on a lectin, for example, a lectin that is free in a solution or a lectin that is displayed on the outer surface of the membrane of a cell.

Lectins include proteins that have binding sites for carbohydrate moieties. For example, lectins can play a role in the immune response of an organism by binding to carbohydrates displayed on the surface of pathogens such as bacteria, parasites, yeasts, and viruses. For example, lectins can play a role in the attachment of bacteria to host cells.

Methods according to the invention include producing and using catanionic vesicles, which are capable of targeted delivery of sequestered or encapsulated contents through specific carbohydrate mediated interactions. Vesicles produced in accordance with this invention can include a mixture of cationic and anionic surfactants, with one or more bioconjugate components. The surfactants can be single-tailed monoalkyl surfactants. As is known in the art, surfactants in general are a broad class of structurally diverse molecules. Surfactants are amphipathic molecules composed of one or more than one hydrophobic hydrocarbon region referred to as the “tail” region, and a hydrophilic, polar region referred to as the “head region” or “head group.” The amphipathic nature of these molecules governs their behavior at and influence upon phase interfaces.

Vesicles have a number of important utilities, including chemical and biochemical applications. Both vesicles and liposomes are of considerable interest in the controlled release and targeted delivery of pharmaceutically active agents in humans, animals, and plants, for example, in the fields of drug delivery, agrochemicals, and cosmetics. For example, vesicles can be useful for the targeted delivery of pesticides, fertilizers, and nutrients in agriculture. For example, loading a medication into a vesicle or liposome can serve to protect the medication from degradation or dilution in the blood and enhance delivery to specific cell types in the body having specific biochemical attributes.

Catanionic surfactant vesicles have several advantages over conventional phospholipid vesicles. For example, they form spontaneously without the need for additional sonication or extrusion, have an extremely long shelf life, and are formed from raw materials that are inexpensive in comparison with synthetic or purified phospholipids. Catanionic vesicles can be spontaneously generated when the individual surfactants are mixed with water in the right proportion. Vesicle formation can be quicker and easier in comparison with phospholipid liposomes, because extrusion or sonication steps are not required. Furthermore, the required materials are common surfactants that are cheaper than purified or synthetic phospholipids. Catanionic vesicles can be stable for very long periods of time, although it is not clear whether catanionic vesicles are truly equilibrium structures.

An embodiment according to the invention makes use of a targeting strategy that naturally occurs in biological systems involving glycosyl-protein and/or glycosyl-glycosyl-mediated recognition. Glycosyl-mediated cell-cell recognition is important, for example, in the infectivity of pathogens, the development of an immune response, and reproduction. The recognition process under these circumstances is fundamentally different than protein-protein or antibody-antigen interactions at cell surfaces in that glycosyl recognition is a multidentate process. Because each binding event of a glycosyl-mediated system involves weak interactions (H-bonding), the receptors involved must establish multiple interactions to achieve high specificity. Thus, the recognition of glycosyl residues on the cell surface requires the clustering of surface receptors. This multidentate binding process of the oligosaccharide-mediated recognition system that is adopted in this invention can, in certain cases, provide advantages over other recognition strategies involving biomolecules such as proteins or nucleic acids. The glycosyl functionalized vesicles described herein are able to present a multidentate display of binding residues, as though they were cells themselves.

In an embodiment according to the present invention, vesicles are prepared in aqueous solution from simple, single-chain surfactants and bioconjugates. The vesicles can contain at least one anionic surfactant, at least one cationic surfactant, and at least one bioconjugate species.

For example, a catanionic vesicle according to the present invention can sequester a solute molecule or solute ion in an inner pool bounded by the inner surface of the bilayer or in the bilayer itself. Such a solute molecule or solute ion can be, for example, a dye, a radionuclide, a pharmaceutical agent, a biotherapeutic agent, a chemotherapeutic agent, a radiotherapeutic agent, a metal, a natural product, a peptide, an oligopeptide, a polypeptide, a saccharide, an oligosaccharide, a polysaccharide, a nucleotide, an oligonucleotide, a polynucleotide, DNA, RNA, carboxyfluoroscein (CF), sulfarhodamine 101 (SR 101), Lucifer yellow (LY), rhodamine 6G (R6G) Doxorubicin, derivatives of these, and combinations.

A derivative of a chemical compound can include, for example, an analog in which one or more atoms of the compound are substituted by other atoms or groups of atoms. For example, a hydrogen may be replaced by a fluorine atom or a methyl group to form a derivative. For example, an oxygen atom may be replaced by a sulfur atom or vice-versa.

For example, catanionic vesicles according to the present invention can include a dye that can be used as a tracer or label, e.g., for research or diagnostic applications. Examples of dyes include carboxyfluoroscein (CF), sulfarhodamine 101 (SR 101), Lucifer yellow (LY), and rhodamine 6G (R6G). A radionuclide can be used as a tracer, e.g., for research or diagnostic applications. For example, the radionuclide can be a positron emitter, useful in positron emission tomography (PET), or a gamma emitter, useful in single photon emission computed tomography (SPECT). A dye or radionuclide used as a tracer can be used to locate regions where a receptor, such as of a lectin, is present that the carbohydrate moiety of a glycoconjugate binds with and targets. For example, the catanionic surfactant vesicle can include a glycoconjugate of which the carbohydrate moiety is selected to target a lectin on a bacterial pathogen, such as Neisseria gonorrhoeae or Francisella tularensis. The catanionic surfactant vesicle can be administered to a sample or a subject and the dye or radionuclide can be imaged to detect the vesicle. Accumulation of the vesicle can indicate the target and an organism presenting the target, for example, a bacterial pathogen. For example, administration of a labeled catanionic surfactant vesicle can be used to diagnose the presence of and locate an infection associated with a pathogen.

A pharmaceutical agent can include, for example, an antibiotic, such as an antibacterial agent, an antiviral agent, or another agent that inhibits, weakens, or kills a pathogen, or otherwise modifies a natural biological process. A biotherapeutic agent can include, for example, a naturally occurring molecule, a molecule derived from a naturally occurring molecule, a molecule that is similar to a naturally occurring molecule, or a molecule that has portions that resemble a naturally occurring biological molecule. For example, a biotherapeutic agent can include a protein, e.g., human growth hormone or insulin, a saccharide, or a nucleotide. For example, a nucleotide may be inserted into a cell as part of a gene therapy treatment. A chemotherapeutic agent can include, for example, a non-selective cytotoxic agent or a selective cytotoxic agent that causes greater damage to cancer cells than to normal cells. Because catanionic vesicles including bioconjugates on the bilayer can target cells, such as cancer cells, non-selective cytotoxic agents can be sequestered in the vesicles, so that the cytotoxic agent is delivered only (or primarily to cancer cells), so that cancer cells are exclusively (or primarily) damaged with no (or minimal) damage to normal cells. Alternatively, a selective cytotoxic agent can be sequestered in a catanionic vesicle including bioconjugates on the bilayer; the targeting of cancer cells can further enhance the selective destruction of cancer cells and sparing of normal cells. In addition to cancer cells, other cells can be targeted, for example, cells infected with a virus or other pathogen and pathogenic bacteria or other pathogenic organisms. Doxorubicin is an example of a chemotherapeutic agent. A radiotherapeutic agent can include, for example, a radionuclide that emits radiation that causes damage to cells. If the emitted radiation is non-selective, that is, causes damage to normal cells as well as cancer cells, the sequestering of the radionuclide in a vesicle that includes bioconjugates to target cancer cells, can impart selectivity to the therapy, in that the vesicles containing the radionuclide will tend to aggregate around cancer as opposed to normal cells, so that cancer cells are preferentially destroyed. The radionuclide can be chosen because it emits radiation that has a short range in an animal, e.g., a human body, for example, because it emits alpha radiation rather than gamma radiation. The short range of the radiation can enhance specificity, in that cell damage is localized to groups of cancer cells, for example, in a tumor. The radionuclide can be chosen for the selectivity of the radiation it emits, for example, because the radiation causes greater damage to cancer cells than to normal cells. The sequestering of such a selective radionuclide in a vesicle that includes bioconjugates that target cancer cells can further enhance the selectivity.

In an embodiment, both a tracer or labeling agent and a therapeutic agent can be sequestered inside a catanionic vesicle including a bioconjugate according to the present invention. Such an approach can be used to simultaneously treat and monitor the progress of treatment of an animal or human. For example, a dye and a pharmaceutical can be sequestered in vesicles containing a glycoconjugate on the surface that binds to lectins on target cells. The pharmaceutical can treat the target cells and the dye can be tracked, e.g., by fluorescence imaging to ensure that the vesicles effectively deliver the dye to the target cell. In some cases, a single compound can serve as both a tracer or label and as a therapeutic agent. For example, a radionuclide can be sequestered in a vesicle, and the bioconjugate on the surface of the vesicle can adhere to a target cell, e.g., a cancer cell, so that the radiation emitted by the decaying radionuclide destroys the cancer cell. The emission of radiation by the radionuclide can be monitored, for example, by an imaging method, to ensure that the vesicles are delivering the radionuclide to the target, e.g., cancer cells, and not to other cells, e.g., normal cells.

### Surfactant Components of Catanionic Surfactant Vesicles

The single-tailed, anionic surfactant can include an amphipathic molecule having a C₆ to C₂₀ hydrocarbon tail region and a hydrophilic, polar head group. The head-group on the anionic surfactant can be, for example, sulfonate, sulfate, carboxylate, benzene sulfonate, or phosphate. The single-tailed, cationic surfactant can include an amphipathic molecule having a C₆ to C₂₀ hydrocarbon tail region and a hydrophilic polar head group. The head group on the cationic surfactant can be, for example, a quaternary ammonium group, a sulfonium group, or a phosphonium group.

The size and curvature properties (shape) of catanionic vesicles formed according to embodiments of the invention can vary depending upon factors such as the length of the hydrocarbon tail regions of the constituent surfactants and the nature of the polar head groups. At a common ˜1% bioconjugate-to-surfactant ratio, the bioconjugate can have no observable effect on vesicle shape, size, or stability in aqueous media. The diameter of vesicles according to the invention can be, for example from about 10 to about 250 nanometers, for example, from about 30 to about 150 nm. The vesicle size can be influenced by selecting the relative lengths of the hydrocarbon tail regions of the anionic and cationic surfactants. For example, large vesicles, e.g., vesicles of from 150 to 200 nanometers diameter, can be formed when there is disparity between the length of the hydrocarbon tail on the anionic surfactant and the hydrocarbon tail on the cationic surfactant. For example, large vesicles can be formed when a C₁₆ cationic surfactant solution is combined with a C₈ anionic surfactant solution. Smaller vesicles can be produced by using anionic and cationic surfactant species of which the lengths of the hydrocarbon tails are more closely matched. The permeability characteristics of vesicles according to the present invention can be influenced by the nature of the constituent surfactants, for example, the chain length of the hydrocarbon tail regions of the surfactants. Longer tail lengths on the surfactant molecules can decrease the permeability of the vesicles by increasing the thickness and hydrophobicity of the vesicle membrane (bilayer). The control of reagent and substrate permeation across vesicle membranes can be an important parameter, for example, when using the vesicles as microreactors.

Exemplary anionic, single-chain surface active agents include alkyl sulfates, alkyl sulfonates, alkyl benzene sulfonates, and saturated or unsaturated fatty acids and their salts. Moieties comprising the polar head group in the cationic surfactant can include, for example, quaternary ammonium, pyridinium, sulfonium, and/or phosphonium groups. For example, the polar head group can include trimethylammonium. Exemplary cationic, single-chain surface active agents include alkyl trimethylammonium halides, alkyl trimethylammonium tosylates, and N-alkyl pyridinium halides.

Alkyl sulfates can include sodium octyl sulfate, sodium decyl sulfate, sodium dodecyl sulfate, and sodium tetra-decyl sulfate. Alkyl sulfonates can include sodium octyl sulfonate, sodium decyl sulfonate, and sodium dodecyl sulfonate. Alkyl benzene sulfonates can include sodium octyl benzene sulfonate, sodium decyl benzene sulfonate, and sodium dodecyl benzene sulfonate. Fatty acid salts can include sodium octanoate, sodium decanoate, sodium dodecanoate, and the sodium salt of oleic acid.

Alkyl trimethylammonium halides can include octyl trimethylammonium bromide, decyl trimethylammonium bromide, dodecyl trimethylammonium bromide, myristyl trimethylammonium bromide, and cetyl trimethylammonium bromide. Alkyl trimethylammonium tosylates can include octyl trimethylammonium tosylate, decyl trimethylammonium tosylate, dodecyl trimethylammonium tosylate, myristyl trimethylammonium tosylate, and cetyl trimethylammonium tosylate. For example, N-alkyl pyridinium halides can include decyl pyridinium chloride, dodecyl pyridinium chloride, cetyl pyridinium chloride, decyl pyridinium bromide, dodecyl pyridinium bromide, cetyl pyridinium bromide, decyl pyridinium iodide, dodecyl pyridinium iodide, cetyl pyridinium iodide.

Surfactants that can be used to form catanionic vesicles according to the present invention include, for example, SDS, DTAC, DTAB, DPC, DDAO, DDAB, SOS, and AOT.

It will be understood that the above listings are representative rather than exhaustive. It will also be appreciated that many surfactants are available as polydisperse mixtures rather than as homogeneous preparations of a single surfactant species and such mixtures are also contemplated by this invention.

### Glycoconjugate Component of Catanionic Surfactant Vesicles

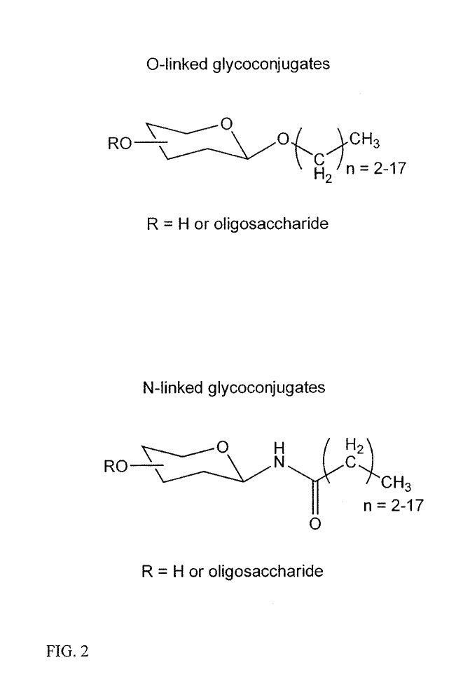

The glycoconjugate component can be generally characterized as a carbohydrate moiety with a hydrophobic group, for example, an alkyl chain, attached. The glycoconjugate can be generated from a wide variety of carbohydrates, and be given various hydrophobic groups, for example, alkyl chains of various lengths. Examples of carbohydrates include lactose, maltose, maltotriose, and glucose, among many others which one skilled in the art will recognize. FIG. 2 presents the chemical structures of some sample glycoconjugates.

Examples of the production of catanionic surfactant vesicles, glycoconjugates, and catanionic surfactant vesicles functionalized with (that is, bearing) glycoconjugates are presented below.

### Bioactivity of Glycoconjugate Bearing Catanionic Surfactant Vesicles

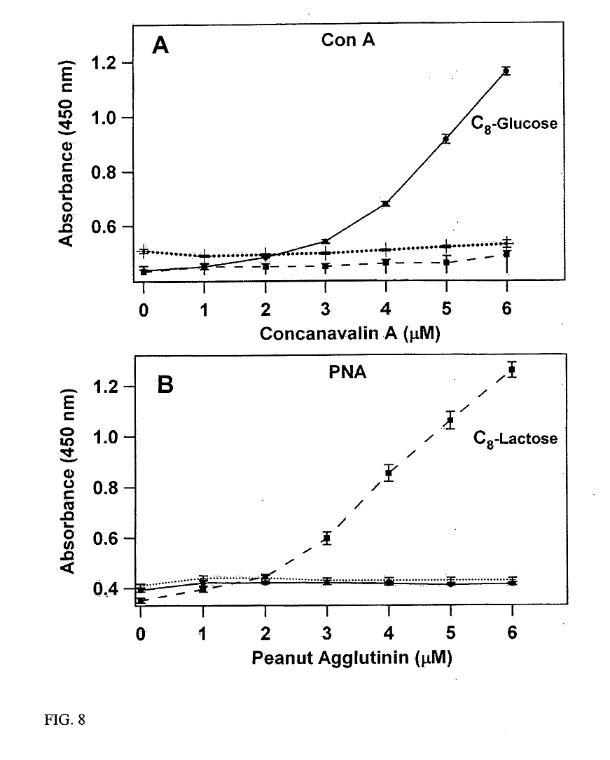

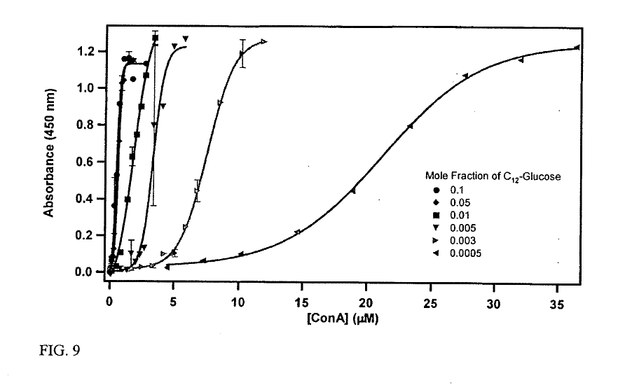

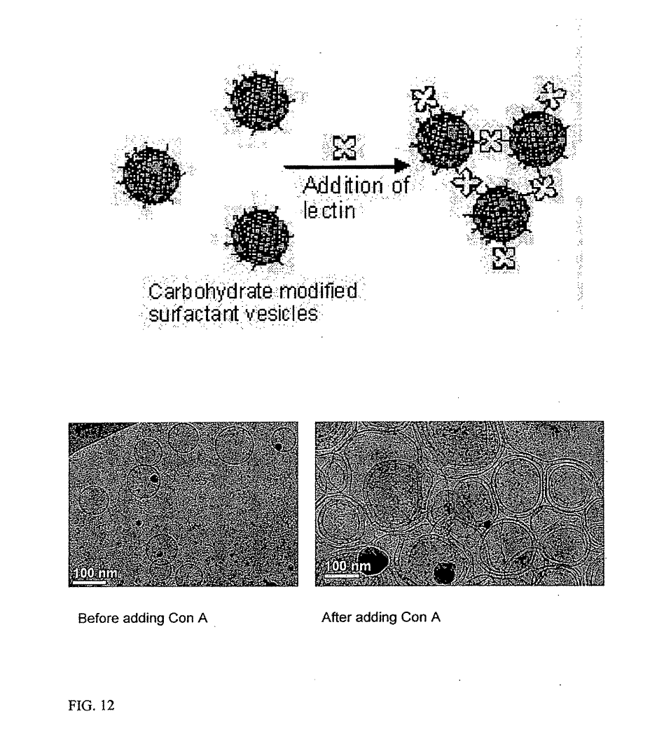

To confirm that the carbohydrates introduced to form the glycoconjugate bearing catanionic surfactant vesicles could serve as targeting entities, their binding to lectins was investigated. Lectins have high binding selectivity for their carbohydrate ligands. Lectins were used to prove that ligands were present and exposed on the vesicle surface. Binding assays were conducted using concanavalin A (Con A) to probe for the presence of surface glucose residues. Con A binds selectively to the monosaccharides mannose and glucose and to polysaccharides with terminal glucose or mannose residues. To test whether the carbohydrate groups located at the exterior of the glycoconjugate bearing catanionic surfactant vesicles are bioactive and not embedded or denatured at the vesicle interface, Con A-induced vesicle aggregation was studied using a turbidity assay. Above pH 7, Con A is a homotetramer and, thus, can bind multiple carbohydrates resulting in aggregation of glucose or mannose bearing vesicles (see FIGS. 8 and 9). Monitoring Con A-induced turbidity provided a convenient method to determine the bioavailability of synthetic mannose or glucose-functionalized glycoconjugates. Turbidity increases in carbohydrate-modified vesicle solutions upon the addition of a multimeric lectin if the lectin recognizes and binds to carbohydrates on different vesicles, as illustrated by FIG. 12.

FIG. 8(a) summarizes results from the Con A aggregation experiments used to test the selectivity of the lectins PNA and Con A for the incorporated glycoconjugates. Bare vesicles and vesicles containing lactose glycoconjugate showed no increase in turbidity when titrated with Con A. Conversely, vesicles carrying the glucose glycoconjugate had a distinct increase in turbidity with increasing additions of Con A above 2.0.mu.M. The increase in turbidity was readily visible by eye and was due to aggregation of vesicles that occurs when a Con A tetramer binds glucose on different vesicles. The ionic strength of the buffer is sufficient to lower the Debye length to less than that of the lectin-tetramer/carbohydrate linkage length (ca. 6 nm), but not high enough to induce spontaneous vesicle aggregation. FIG. 8(b) shows an analogous set of experiments using the lectin peanut agglutinin (PNA). PNA has monosaccharide binding selectivity for galactose and is also homotetrameric at physiological pH. As with Con A, the solution turbidity for the three vesicle samples was monitored with increasing PNA concentration. In the case of PNA, an increase in turbidity was observed only in the presence of C₈-lactose modified vesicles. Binding of PNA to the terminal galactose of the lactose glycoconjugate induces agglutination in the C₈-lactose bearing vesicles. Control experiments using solutions of only glycoconjugates, and no vesicles, gave no change in turbidity with addition of lectins (data not shown). The results outlined in FIGS. 8a and 8b suggests that amphiphilic glycoconjugates can be used to functionalize surfactant vesicles for recognition by cell surface receptors and represents a promising first step toward targeted delivery using carbohydrate-functionalized surfactant vesicles.

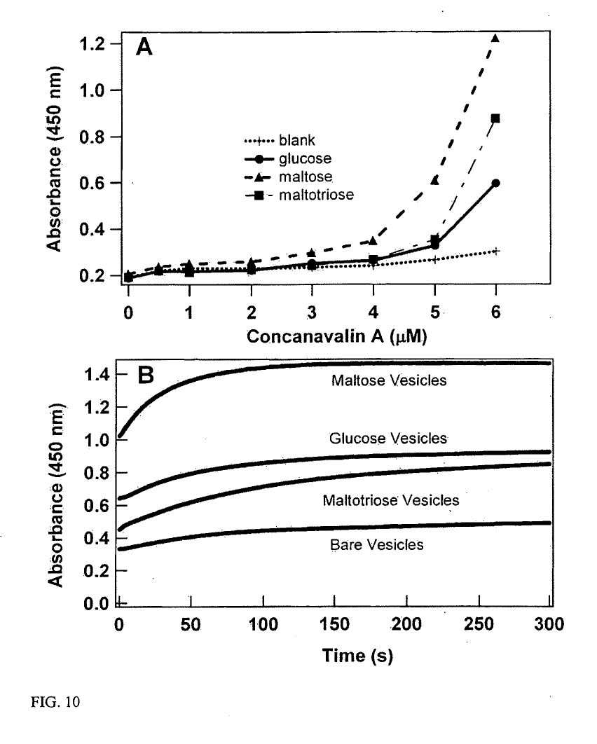

In FIGS. 8a and 8b, turbidity is shown to increase slightly more rapidly with PNA binding to lactose-modified vesicles than with Con A binding to glucose-modified vesicles. Without being bound by theory, this difference in agglutination is rationalized by assuming increased accessibility at the bilayer interface of the terminal galactose in the disaccharide lactose relative to the monosaccharide glucose. Others have demonstrated the binding of Con A to glycolipids embedded in phospholipid vesicle membranes and have shown that the inclusion of a water soluble spacer group between the alkyl chains and the carbohydrate head group improves binding. To explore the effect of oligosaccharide length on lectin-induced agglutination, vesicles were prepared with three different glucose-containing glycoconjugates: C₈-glucose, C₈-maltose and C₈-maltotriose (FIG. 10) and their aggregation as a function of Con A concentration was measured. FIG. 10 summarizes the results from these experiments. The maltose-conjugate, a disaccharide, shows increased turbidity relative to C₈-glucose, the monosaccharide analog, indicating stronger binding by the lectin.

Agglutination experiments with C₁₂-glucose coated vesicles and Con A were conducted. Turbidity as a function of time was monitored and the initial rate was used to evaluate the multidentate nature of Con A—glucose interactions at the bilayer interface. This method provides a facile path to evaluating lectin structure as described below.

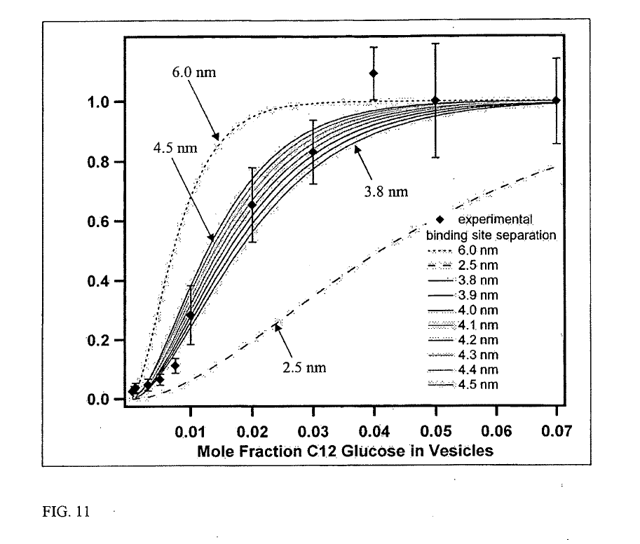

The buffered Con A as described in the turbidity assay was used. The absorbance at 490 nm was monitored with time for the reaction of buffered Con A with vesicles conjugated with different mole fractions of C₁₂-glu. A blank containing equal parts vesicle samples and buffer with no Con A was used. Each run was performed by first adding 250 μL of vesicle sample to the cuvette, then placing the cuvette in the UV-Vis instrument, adding 250 μL of buffered Con A, then immediately starting acquisition of the kinetics data. The concentration of Con A used was 2.5 μM. For each kinetics run, the initial rate was found from the slope of the initial linear region of absorbance plot. The rates were plotted versus the mole fraction of C₁₂-glucose in the corresponding vesicle sample to obtain FIG. 11.

FIG. 11 presents the initial rate for aggregation over a range of C₁₂-glucose surface density. The initial rate of aggregation is directly proportional to the rate of Con A binding at the vesicle interface and shows an interesting trend. At low mole fractions up to 0.01 the rate increases linearly with C₁₂-glucose mole fraction and then undergoes a sharp increase in slope before leveling off above 0.03. The initial binding rate will be the product of ConA-vesicle collision frequency (v_{coll}) and a probability factor (φ) which accounts for factors such as orientation, kinetic energy and ligand density.

Rate∝v_{coll}φ (Equation 1)

In our experiments the Con A and vesicle concentrations are constant and therefore the variation in initial rate must be due to the factor φ. This variation can be captured by a simple model based on multivalent interactions which assumes the following: i) non-interacting randomly distributed ligands on the membrane surface; ii) an effective sampling area by the Con A tetramer during a collision with the membrane surface; and iii) the presence of two ligands in the effective area. The first criterion, non-interacting ligands, invokes the Poisson distribution to describe the ligand distribution. The average effective separation between binding sites in ConA can be used to estimate the effective sampling area. This concept has been invoked to describe the binding of the enzyme carbonic anhydrase to target substrates of varying ligand density. The third criterion is supported by the fact that Con A has a significantly higher K_{d} value for multivalent as compared to monovalent ligands. To induce aggregation, the ConA tetramer must bind two glycosyl residues in order for a protein-vesicle collision to result in persistent binding. Using an approach by Walker and Zasadzinski we obtained the average area occupied by a single noninteracting glucose residue (ρ) as

$$ρ = \frac{{X}_{glu}}{0.48 {nm}^{2}} . \qquad ( Equation 2 )$$

The value of ρ can be used to determine the probability of a Con A tetramer encountering more than two residues in a single collision. The model assumes that Con A collides and binds with the first residue and prior to dissociation it sweeps-out an effective target area (A=πd²) on the bilayer surface determined by the effective binding site separation distance of the tetramer d. The average number of ligands encountered per collision will then be

μ=ρA. (Equation 3)

The probability that a ConA tetramer colliding with the exterior bilayer will encounter two or more glycosyl residues, based on a Poisson distribution of glycosyl sites, is

$$P ( N \geq 2 ) = {\sum }_{N = 2}^{∞} \frac{{μ}^{N}}{N !} {e}^{- μ} . \qquad ( Equation 4 )$$

Equation 4 gives the probability that two or more residues will be found in the effective target area, where N is the number of occurrences of a residue in the effective target area described by the binding site separation distance.

FIG. 11 presents a range of simulated curves generated using Equations 1-4 with different assumed values of d. The two extreme curves are for the limits of the literature values for d. The eight central curves model values of d from 3.8 to 4.5 nm in increments of 0.1 nm. The best fit based on a chi-squared analysis is given by the curve corresponding to 4.3 nm. Thus, the data obtained suggest an effective binding site separation distance of 4.3 nm for ConA, well within the literature values. This modeling also provides a good description of the observed kinetic trend. When the ligand concentration is such that the average separation of accessible glucose residues is larger than the separation of saccharide binding sites on the Con A tetramer, the rate of binding depends linearly on C₁₂-glucose concentration. However, if the ligand concentration is at a point where the average separation of glucose ligands is smaller than the saccharide binding site separation, then the rate of agglutination is zeroth order with respect to C₁₂-glucose. At this point the ligand density is approximately 0.083 residues/nm². At this density the rate of binding becomes saturated and independent of the glucose surface coverage. This model assumes no clustering of ligands, and the good fit to the data suggests that ligand clustering does not play a role in this system previously observed.

In summary, the above represents a new method and corresponding theoretical description for measuring the binding dependence of Con A on ligand density at to an anionic membrane interface. The consistency of the literature value for Con A binding site separation distance with the value used for optimization of the Poisson analysis strengthens the support for this model and suggests a possible novel method for predicting the binding site separation of lectins. Additional studies are in progress to obtain analogous data using other lectin/carbohydrate pairs. These results will determine the viability of this novel method for predicting the distance between saccharide binding sites on other lectins.

The ability to create glycoconjugates that bind to specific cellular receptors, and integrate those conjugates into catanionic vesicles can have important utility in fields such as medicine, pharmacology, agriculture, and veterinary medicine.

### Example 1

### Applications

In a method according to this invention, cancer in an animal can be treated by destroying cancerous cells. Such a method can include administering a bioconjugate functionalized catanionic vesicle to the animal, the catanionic vesicle including a chemotherapeutic or radiotherapeutic agent, and the surface of the vesicle including one or more conjugated sugar groups that bind to receptors on the cancerous cells, so that the administered vesicles interact specifically with cancerous cells.

In a method according to the invention, an infectious disease in an animal can be treated by destroying a microbe. Such a method can include administering a bioconjugate functionalized catanionic vesicle to the animal, the catanionic vesicle including an antimicrobial agent, and the surface of the vesicle including one or more bioconjugate (in the case of a glycoconjugate, a sugar conjugated) groups that bind to receptors on the microbe, so that the vesicles specifically interact with the microbe.

In a method according to the invention, cancer in an animal can be located and diagnosed. Such a method can include administering a bioconjugate functionalized catanionic vesicle to the animal, the catanionic vesicle including a dye, and the surface of the vesicle including one or more bioconjugate (in the case of a glycoconjugate, conjugated sugar groups) groups that bind to receptors on cancerous cells comprising the cancer, so that the vesicles specifically interact with the cancer cells, thereby placing the dye in physical proximity to the cancer cells for enhanced detection and location.

In a method according to this invention, gene therapy can be administered to target cells of an animal. Gene therapy includes the insertion of a nucleic acid into a cell to change the genetic instruction set of the cell. Gene therapy can be used to treat diseases, for example, hereditary diseases. A method according to the invention can include administering a bioconjugate functionalized catanionic vesicle to the animal, the catanionic vesicle comprising a nucleotide sequence that induces the gene therapy, and the surface of the vesicle comprising one or more conjugated sugar groups that bind to receptors on the target cells, so that the vesicles specifically interact with the targeted cells in order to specifically deliver the nucleotide sequence to the cell.

### Example 2

### Formation of Catanionic Surfactant Vesicles

The surfactants CTAT, SDBS, and Triton X-100 were purchased from Aldrich Chemicals. The fluorescent dyes CF, sulforhodamine 101 (SR 101), and Lucifer yellow (LY) were purchased from Molecular Probes, while the dye rhodamine 6G (R6G) and the chemotherapeutic drug, doxorubicin hydrochloride (Dox) were purchased from Fluka. All materials were used without further purification. The dry surfactants, CTAT and SDBS, were stored in a desiccator to prevent water absorption.

Vesicle samples were prepared at two different surfactant compositions, 7:3 and 3:7 w/w CTAT to SDBS, which are denoted as V⁺ and V⁻, respectively. V⁺ refers to the excess positive charge on the vesicle bilayers when there is an excess of CTAT, and likewise, V⁻ refers to vesicles with a net negative charge due to an excess of SDBS. All samples were prepared at a total surfactant concentration of 1 wt. %. The surfactants were weighed and mixed with deionized water by gentle stirring, and then allowed to equilibrate at room temperature for at least 48 h.

Vesicle sizes in solution were monitored using dynamic light scattering (DLS) on a Photocor-FC instrument. The light source was a 5 mW laser at 633 nm and the scattering angle was 90°. A logarithmic correlator was used to obtain the autocorrelation function, which was analyzed by the method of cumulants to yield a diffusion coefficient. The apparent hydrodynamic size of the vesicles was obtained from the diffusion coefficient through the Stokes-Einstein relationship. The intensity (total counts) of the signal was also recorded for each sample.

Small angle neutron scattering (SANS) experiments were conducted on the neat vesicles as well as vesicle-solute mixtures to probe whether there were any changes in vesicle size or bilayer integrity caused by the solutes. All samples for SANS experiments were prepared using deuterium oxide (99% D, from Cambridge Isotopes) in place of water. The measurements were made on the NG-7 (30 m) beamline at NIST in Gaithersburg, Md. Neutrons with a wavelength of 6 Å were selected. Two sample-detector distances of 1.33 m and 13.2 m were used to probe a wide range of wave vectors from 0.004-0.4 Å⁻¹. Samples were studied in 2 mm quartz cells at 25° C. The scattering spectra were corrected and placed on an absolute scale using calibration standards provided by NIST.

### Example 3

### Production of Glycoconjugates

In an embodiment, glycoconjugates are produced using the following generalized procedure (see FIG. 13). A carbohydrate peracetate is generated from a carbohydrate treated with NaOAc in acetic anhydride. The peracetate solution is treated with trimethylsilyl azide followed by a solution of SnCl₄ to generate a glycosyl azide. Then, the glycosyl azide is converted to an acylated glycoconjugate through treatment with diisopropylethylamine followed by a solution of PMe₃, after which a fatty acid (such as octanoic acid) is added. The final glycoconjugate is produced by reacting the acylated glycoconjugate with sodium methoxide. The length of the alkyl chain on the final glycoconjugate is determined by the nature of the fatty acid. For example, octanoic acid yields a C₈ chain, whereas dodecanoic (lauric) acid yields a C₁₂ chain.

Steps in a glycoconjugate synthesis are outlined below:

1. To a refluxing suspension of anhydrous NaOAc (4.0 equiv) in acetic anhydride (20 equiv) add carbohydrate (1.0 equiv). Reflux the reaction mixture for 3 h and cool to 100° C., then immediately transfer into ice-water mixture and stir vigorously until forming a gum. After decanting with water, dissolve the gum in CH₂Cl₂ and then wash with sat. aq. NaHCO₃, H₂O, dry over MgSO₄, filter, and concentrate in vacuo. Purify the crude product by column chromatography to give β-glycosyl peracetate.
2. To a solution of glycosyl peracetate (1.0 equiv) in anhydrous CH₂Cl₂ add trimethylsilyl azide (1.3 equiv), followed by 1.0 M solution of SnCl₄ (0.5 equiv). Stir the resulting solution at room temperature for 24 h under a nitrogen atmosphere. Dilute the reaction mixture with CH₂Cl₂, wash with sat, aq. NaHCO₃, H₂O, dry over MgSO₄, filter, and concentrate in vacuo. Purify the crude product either by column chromatography or recrystallization to give β-glycosyl azide.
3. To a solution of glycosyl azide (1.0 equiv) in anhydrous CH₂Cl₂ add diisopropylethylamine (2.0 equiv), followed by 1.0 M solution of PMe₃ (1.2 equiv). Stir the reaction mixture at room temperature for 30 min under a nitrogen atmosphere and then add octanoic acid (2.0 equiv). After stirring for 24 h, dilute the reaction mixture with CH₂Cl₂ and wash with brine, dry over MgSO₄, filter, and concentrate in vacuo. Purify the crude product by column chromatography to give acetylated β-glycoconjugate with trace α-anomer.
4. To a solution of acetylated glycoconjugate (1.0 equiv) in MeOH add 0.2 M solution of sodium methoxide (given equiv) and then stir at room temperature for 24 h under a nitrogen atmosphere. Neutralize the reaction mixture with Dowex MAC-3 resin (weakly acidic cation exchanger), filter, and concentrate in vacuo. Purify the crude product by short column chromatography to give .beta.-glycoconjugate with trace .alpha.-anomer.

Glycoconjugates can be formed from single saccharides, oligosaccharides, or polysaccharides.

### Example 4

### Production of Peptidoconjugates

Peptidoconjugates were prepared by the reaction of a peptide with the N-hydroxysuccinimide ester of octanoic acid (C₈ acid) in aqueous acetone. For example, 1.5 mg of PADRE dissolved in 10 mL of 0.1 M HEPES buffer at pH 7.4 was treated with a solution of 0.5 mg of the N-hydroxysuccinimide ester of octanoic acid in 1.0 mL of acetone at room temperature for 24 hours. The peptidoconjugate was isolated by extraction of the reaction mixture with ethyl acetate, followed by acidification of the aqueous layer to pH 3, a second ethyl acetate extraction, and finally, adjustment of the pH of the aqueous layer to 7.0.

The method of preparing a peptidoconjugate can depend on the specific peptide sequence to be conjugated, as will be appreciated by one skilled in the art. The tertiary structure of a peptide can be important for it to have a desired biological effect (e.g., stimulation of an immune response or binding to a cell surface). Moreover, it can be important for a particular feature on a folded peptide, e.g., a cleft or a salient region, to be presented to have the desired biological effect. One of skill in the art will consider such factors in designing the structure of a peptidoconjugate and designing a method for the synthesis of a peptidoconjugate. For example, the peptide sequence can be linked to a hydrophobic group at its N-terminus, at its C-terminus, or at an intermediary amino acid to form a peptidoconjugate.

Peptidoconjugates can be formed from single amino acids, oligopeptides, or polypeptides.

### Example 5

Production of Glycoconjugate and/or Peptidoconjugate Functionalized Catanionic Surfactant Vesicles

Unilamellar vesicles can be formed spontaneously by combining an aqueous to solution of a single-tailed, anionic surfactant with an aqueous solution of a single-tailed, cationic surfactant. The resulting catanionic vesicles appear to be equilibrium vesicles, i.e., they can be stable over extended time periods, such as up to one year. Catanionic vesicles so prepared can be capable of withstanding freeze-thaw cycles without disruption or release of their contents.

In a method according to the invention, glycoconjugate functionalized (that is, glycoconjugate bearing) catanionic surfactant vesicles are spontaneously formed by mixing anionic and cationic surfactants in an aqueous solution of glycoconjugates. The surfactants can be mixed into solution either as dry chemicals, or as aqueous solutions. The vesicles form without the need for mechanical or chemical treatments beyond mild stirring to aid in mixing and dissolving the two surfactants. When formed in this manner, the carbohydrate portion of the glycoconjugate's location is understood to be distributed equally between the internal and external leaves of the vesicle membrane.

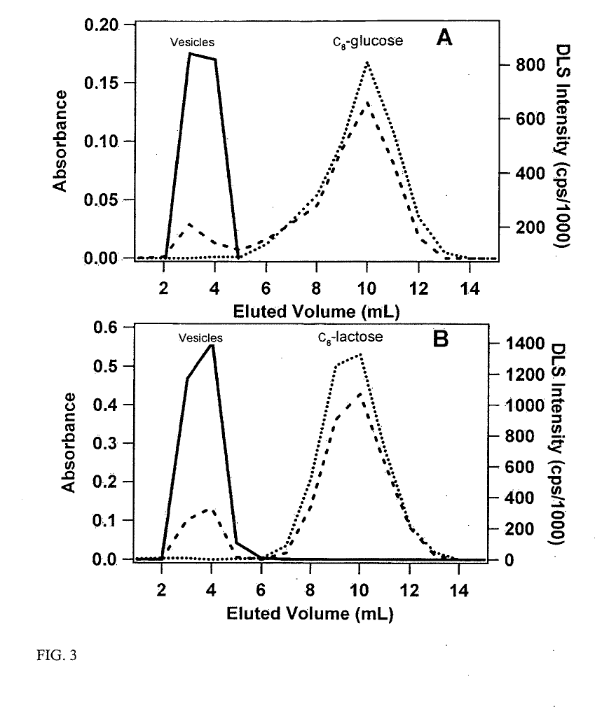

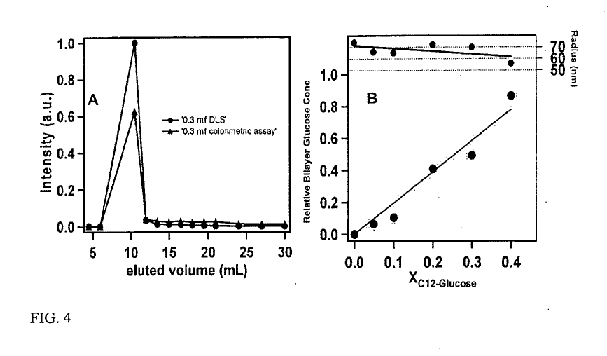

In an alternative method according to the invention, glycoconjugate functionalized vesicles can be generated by pre-forming a solution of catanionic vesicles without the glycoconjugates, then adding a solution of glycoconjugates. When formed in this manner, the carbohydrate portion of the glycoconjugate's location is understood to be distributed only on the external leaves of the vesicle membrane, because the inner leaves are enclosed, i.e., the inner leaves bound the inner pool and do not face the external environment. In either method of preparation, the glycoconjugates are spontaneously incorporated into the vesicles (see FIGS. 3 and 4).

In either method, vesicles containing the glycoconjugate can be concentrated by techniques such as centrifugation or filtration. Vesicles containing glycoconjugates can be separated from unincorporated glycoconjugates and vesicles which have not incorporated glycoconjugates through size exclusion or affinity chromatography (see FIG. 3). The skilled practitioner will realize that there are many other possible techniques for concentration and separation. Additionally, in either of the above methods, the net charge of the vesicles can be selectively modified by altering the ratio of cationic surfactant to anionic surfactant.

In an experiment, glycoconjugates consisting of eight carbon tails were used. In all SDBS-rich vesicle test cases 18-25% of the conjugate eluted with the vesicle fractions. The incorporation values for several different glycoconjugates in SDBS-rich vesicles are shown in Table 1. At the incorporation levels summarized in Table 1, the ratio of carbohydrate conjugate to surfactant is approximately 1:100. At this concentration, vesicle formation is uninhibited and the carbohydrate groups are displayed on the vesicle outer surface. DLS (dynamic light scattering) measurements showed that the vesicle size and sample polydispersity were not significantly affected by inclusion of the glycoconjugate at these levels in SDBS-rich samples (see Table 1). The fact that not all of the glycoconjugate was incorporated suggests an equilibrium between membrane-associated and free glycoconjugate.

TABLE 1

| | Incorporation | Vesicle | Polydispersity |
|---|---|---|---|
| Glycoconjugate | (%)^{a} | Radius^{b} | Index^{b} |
| Bare Vesicle | — | 69 | 0.55 |
| C8-glucose | 18 | 81 | 0.51 |
| C8-lactose | 23 | 68 | 0.48 |
| C8-maltose | 25 | 70 | 0.53 |
| C8-maltotriose | 19 | 58 | 0.55 |

^{a}Incorporation percentage is the fraction of a 1 mM solution of glycoconjugate that elutes with vesicles during SEC
^{b}Hydrodynamic radii and polydispersity index were determined by DLS prior to SEC, see text for details.

In another experiment, a glucose glycoconjugate with a twelve carbon tail, n-dodecyl-β-D-glucopyranoside (C₁₂-glucose), was used. Incorporation studies with this material show that up to 40 mole percent of the vesicle bilayer can be composed of the glycoconjugate (FIG. 4). When the carbon tail length is increased from 8 to 12, incorporation is much higher and vesicles are readily prepared with C₁₂-glucose concentrations up to 40 mole percent. The method for preparing these vesicles is now described. Vesicles were prepared with the surfactants SDBS, CTAT, and C₁₂-glu. Millipore water (18 MΩ) was added to dry surfactants and then stirred for at least 2 h. Total surfactant concentration was kept constant at ˜27 mM. The mole ratio of SDBS to CTAT was 3:1 in all cases (70:30 w/w). For vesicle samples containing different mole fractions of C.sub.12-glu, the amount of SDBS and CTAT was adjusted accordingly to keep the stated ratio of ionic surfactants constant. After stirring, the samples were allowed to equilibrate in the dark at room temperature for at least 48 h. Samples were then passed through a 0.45 μm syringe filter to remove impurity particles such as dust.

Peptidoconjugate functionalized catanionic surfactant vesicles can be formed in a similar manner as glycoconjugate functionalized catanionic surfactant vesicles. For example, peptidoconjugate functionalized (that is, peptidoconjugate bearing) catanionic surfactant vesicles can be spontaneously formed by mixing anionic and cationic surfactants in an aqueous solution of peptidoconjugates. The surfactants can be mixed into solution either as dry chemicals, or as aqueous solutions. The vesicles form without the need for mechanical or chemical treatments beyond mild stirring to aid in mixing and dissolving the two surfactants. When formed in this manner, the peptide portion of the peptidoconjugate's location is understood to be distributed equally between the internal and external leaves of the vesicle membrane. In an alternative method according to the invention, peptidoconjugate functionalized vesicles can be generated by pre-forming a solution of catanionic vesicles without the peptidoconjugates, then adding a solution of peptidoconjugates. When formed in this manner, the peptide portion of the peptidoconjugates location is understood to be distributed only on the external leaves of the vesicle membrane, because the inner leaves are enclosed, i.e., the inner leaves bound the inner pool and do not face the external environment. In either method of preparation, the peptidoconjugates are spontaneously incorporated into the vesicles (see FIGS. 3 and 4).

### Example 6

### Cell Targeting by Catanionic Vesicles Bearing Glycoconjugates

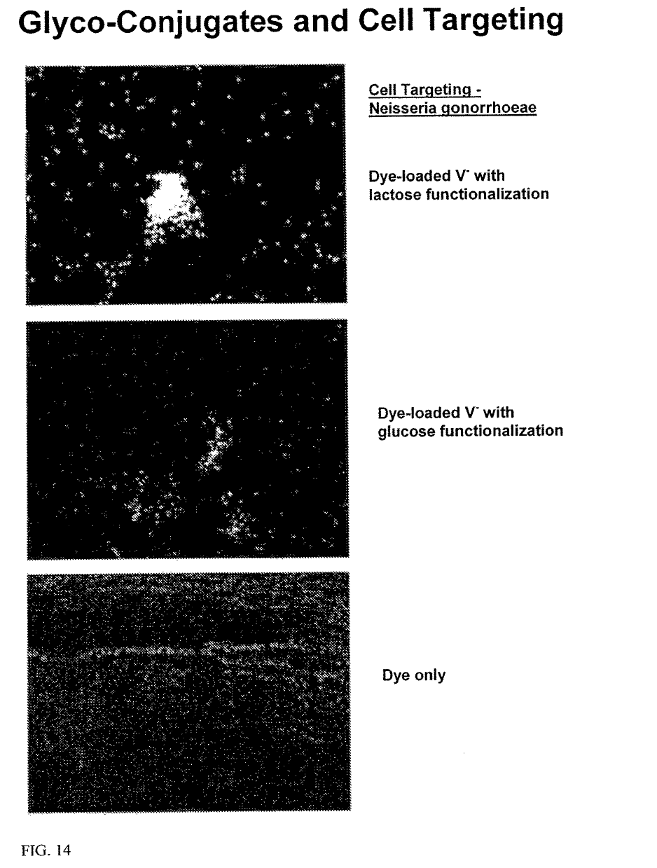

In an embodiment, catanionic vesicles that bear a glycoconjugate with a carbohydrate moiety of the glycoconjugate displayed on the outer surface of the vesicle bilayer were loaded with a fluorescent dye and used in a cell targeting study. A first set of is dye loaded vesicles functionalized with a lactose glycoconjugate were administered to Neisseria gonorrhoeae cells, as shown in FIG. 14. A second set of dye loaded vesicles functionalized with a glucose glycoconjugate were administered to Neisseria gonorrhoeae cells. A third set of dye loaded vesicles were not functionalized with a glycoconjugate. As shown by FIG. 14, the lactose functionalized vesicles adhered to the Neisseria gonorrhoeae cells, as indicated by the fluorescence. By contrast, the vesicles not functionalized with a glycoconjugate did not adhere to the Neisseria gonorrhoeae cells, as indicated by the lack of fluorescence.

### Example 7

### Libraries of Catanionic Vesicles Bearing Bioconjugates

In an embodiment, catanionic vesicles that include a bioconjugate with a carbohydrate and/or peptide moiety of the bioconjugate displayed on the outer surface of the vesicle bilayer can be used as components of a library. For example, such a library can include a first catanionic vesicle with a bioconjugate having a first carbohydrate moiety and a second catanionic vesicle with a bioconjugate having a second carbohydrate moiety different from the first carbohydrate moiety. Such a library can be used for research or diagnostic purposes. For example, a library can include two or more types of catanionic vesicles, each incorporating a bioconjugate, so that a carbohydrate and/or peptide moiety is displayed on the outer surface of the membrane of the vesicle, the different types of catanionic vesicles displaying different carbohydrate and/or peptide moieties. Each different type of catanionic vesicle can further include a label or tracer molecule different from the label or tracer molecule included in different catanionic vesicles.

In a research or diagnostic procedure, such a library including two or more, for example, many, types of catanionic vesicles can be administered to a sample or to a patient. Each carbohydrate and/or peptide moiety can be selected for its specific binding to a receptor site, for example, to a carbohydrate binding site on a lectin, of interest. Because the label or tracer molecule of a given type of catanionic vesicle displaying a certain carbohydrate and/or peptide is known, by identifying the label or tracer molecule retained in a region of a sample or patient, the type of receptor site in that region can be identified. Conclusions about the presence of certain cells, e.g., of pathogenic organisms such as a pathogenic bacterium, that are known to present the identified receptor or the presence of certain substances, for example, a lectin, such as ricin, can then be drawn. For example, a library can include a first type of catanionic vesicle can display a first carbohydrate and/or protein moiety and sequester a first dye that fluoresces at a first wavelength (i.e., fluoresces with a first color, e.g., red) and a second type of catanionic vesicle can display a second carbohydrate and/or protein moiety and sequester a second dye that fluoresces at a second wavelength (i.e., fluoresces with a second color, e.g., green).

### Example 8

### Catanionic Vesicles Bearing Glycoconjugates for Blood Typing Systems

For example, a carbohydrate moiety of a first type of catanionic vesicle in a library can bind with an antibody of the A blood type antigen (anti-A), and a carbohydrate moiety of a second type of catanionic vesicle in a library can bind with an antibody of the B blood type antigen (anti-B). The library can be applied to a blood sample. Agglutination of the first type of catanionic vesicle, for example, can reduce the amount of detected fluorescence of the first color remaining in solution, e.g., red, associated with the first dye, and can indicate the presence of anti-A antibody in serum. Agglutination of the second type of catanionic vesicle, for example, can reduce the amount of detected by fluorescence of the second color remaining in solution, e.g., green, associated with the second dye, and can indicate presence of anti-B antibody in serum. The identification of which type(s) of catanionic vesicle agglutinates by measuring the fluorescence remaining in supernatants can be used in a system or kit for a rapid blood typing procedure. For example, the presence of both anti-A and anti-B can indicate an 0 blood type, the presence of anti-A alone can indicate a B blood type, the presence of anti-B alone can indicate an A blood type, and the presence of neither anti-A nor anti-B can indicate an AB blood type. Additional types of catanionic vesicles with different carbohydrate moieties on their glycoconjugates that bind to other blood-type antibodies can be included in such a library for a blood-typing system or kit.

### Example 9

### Catanionic Vesicles Bearing Glycoconjugates for Lectin Detection Systems

For example, the first carbohydrate moiety of a first type of catanionic vesicle can be selected to bind with a first lectin, and the second carbohydrate moiety of a second type of catanionic vesicle can be selected to bind with a second lectin. For example, such a library can be used to detect whether a first lectin, a second lectin, both, or neither are present. For example, such a library can be used as part of a biothreat detection system, e.g., to detect for the presence of a lectin toxin, such as ricin or abrin. For example, a device can include a component that introduces a sample, e.g., an airborne sample, into solution. A library of catanionic vesicles bearing glycoconjugates can then be introduced into the solution for detection of a lectin in a manner similar to that described for a blood typing system, above.

### Example 10

### Bio-Functionalized Catanionic Surfactant Vesicles as Vaccines

In an embodiment, catanionic vesicles that include a glycoconjugate having a carbohydrate moiety and a hydrophobic group, at least a portion of the hydrophobic group within the bilayer of the vesicle and the carbohydrate moiety on the outside of the vesicle, can be included in a vaccine. Alternatively, the glycoconjugate can be another type of bioconjugate. A bioconjugate can be a glycoconjugate, a peptidoconjugate, or a conjugate having both glyco and peptido groups. Thus, a bioconjugate can have a carbohydrate and/or peptide moiety and a hydrophobic group.

The carbohydrate moiety and/or the peptide moiety can be selected to stimulate an immune response. For example, the carbohydrate moiety can be selected to be the same as, similar to, or the same or similar to a portion of a carbohydrate presented on the surface of a pathogen, such as a bacterium, against which an immune response is to be induced. Because a large number of glycoconjugates can be incorporated into the bilayer of the catanionic vesicle, multiple carbohydrate moieties can be simultaneously presented to immune receptors to elicit an immune response. A vesicle can include more than one type of glycoconjugate or peptidoconjugate, so that more than type of carbohydrate or peptide moiety is presented for the elicitation of an immune response. For example, a vesicle can include glycoconjugates and peptidoconjugates. The peptidoconjugate can be derived from an immunostimulatory peptide, for example, PADRE.

In an experiment, catanionic vesicles formed of SDBS and CTAT were prepared in buffer in the presence of a mixture of the lipid oligosaccharide (LOS) from Neisseria gonorrhoeae (5%-20% mole fraction w/w with total surfactant) (see, J Biol. Chem. 266(29) (1991 Oct. 15) pp. 19303-11) and a C₈-lipid conjugate (1 mole fraction w/w with total surfactant) of an immunogenic peptide, PADRE (Pan-DR T helper cell epitopes). It will be understood that other immunogenic peptides can also be used. The vesicles were prepared from 14 mg of SDBS, 6 mg of CTAT, 1 mg of Neisseria LOS, and 0.1 mg of peptide conjugate in 10 mL of buffer using the standard technology. The resulting vesicles were “anionic” since they contain an excess of the anionic surfactant SDBS. The resulting vesicles were shown to contain both LOS and peptide conjugate by chemical analysis. Inoculation of mice with the modified surfactant vesicles resulted in a strong immune response and antibody production. The antibody titer from the surfactant vaccinated mice was different in both magnitude and type of antibody produced (IgG vs. IgM) compared with mice inoculated with LOS only.

In the experiment, the total Neisseria gonorrhoeae LOS administered to each mouse was 20 μg. The LOS constituted 1% of the weight of the vesicles, the anionic and cationic surfactants accounting for the remaining 99%. The mice used in the experiment averaged about 25 grams in weight.

In an experiment, catanionic vesicles incorporating lipopolysaccharide (LPS) from Francisella tularensis LVS (Live Vaccine Strain) and peptide conjugate were prepared and characterized. Mice inoculated with the LPS- and peptide-functionalized vesicles (5 mice per group at two concentrations) or LPS-functionalized vesicles (no peptide; 5 mice per group) did not become ill, and all survived a challenge with live bacteria. By contrast, mice inoculated with saline alone became visibly ill and only 3/5 mice survived a challenge with bacteria.

In an experiment, the total Francisella tularensis LPS administered to a first set of mice was 2 μg, and the total Francisella tularensis LPS administered to a second set of mice was 0.2 μg. The LOS constituted 1% of the weight of the vesicles, the anionic and cationic surfactants accounting for the remaining 99%. The mice used in the experiment averaged about 25 grams in weight.

It is appreciated that the effective dose(s) of catanionic vesicles and/or agents incorporated in catanionic vesicles administered to treat a condition may vary depending on the patient's age, sex, physical condition, duration and severity of symptoms, nature, duration and severity of the underlying disease or disorder if any, and responsiveness to the administered compound.

For example, to achieve immunoprotection in a human or animal, catanionic vesicles bearing an immune response stimulating agent (e.g., lipid oligosaccharide and/or lipopolysaccharide) can be administered. For example, the immune response stimulating agent can be administered in a dose sufficient to obtain blood concentrations of from about 0.01 μg/ml to about 100 μg/ml; for example, the dose administered can be sufficient to obtain blood concentrations of from about 0.1 μg/ml to about 10 μg/ml; for example, the immune response stimulating agent can be administered in a dose sufficient to obtain a blood concentration of about 1 μg/ml.

The invention includes a vaccine formulation comprising catanionic vesicles including bioconjugates administered in an amount effective to have an immunoprotective effect. For example, the formulation can be administered orally or intravenously. For example, doses of the immune response stimulating agent (e.g., lipid oligosaccharide and/or lipopolysaccharide) in the range of from about 0.001 to about 10 mg/kg body weight can be administered; for example, doses in the range of from about 0.01 to about 1 mg/kg body weight can be administered; for example, a dose of about 0.1 mg/kg body weight can be administered.

For therapies in which the catanionic vesicles convey a therapeutic agent, for example, a pharmaceutical, a chemotherapeutic agent, and/or a radiotherapeutic agent, to cells to be treated, the dosing of the catanionic vesicles can be guided by knowledge of the pharmacological effects of the therapeutic agent as known to one of skill in the art.

For example, the chemotherapeutic agent doxyrubicin can be administered to a human or animal subject in a dose sufficient to achieve a weight of agent per unit body surface area of the subject in a range of from about 0.02 to about 200 mg/m² of body surface area per day; for example in a range of from about 0.2 to about 20 mg/m² of body surface area per day; for example, about 2 mg/m² of body surface area per day. For example, doxyrubicin can be administered in a dose of 20 mg/m² of body surface area once per month.

If the condition of the recipient so requires, the doses may be administered as a continuous or pulsatile infusion. The duration of a treatment may be decades, years, months, weeks, or days, as long as the benefits persist. The foregoing ranges are provided only as guidelines and subject to optimization.

The mode of administration and dosage forms is closely related to the therapeutic amounts of the compounds or compositions which are desirable and efficacious for the given treatment application. Suitable dosage forms include but are not limited to oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. The pharmaceutical composition of the present invention can be administered orally in the form of tablets, pills, capsules, caplets, powders, granules, suspension, gels and the like. Oral compositions can include standard vehicles, excipients, and diluents. The oral dosage forms of the present pharmaceutical composition can be prepared by techniques known in the art and contain a therapeutically effective amount of the catanionic vesicles bearing bioconjugates for the stimulation of an immune response or carrying a therapeutic agent according to the present invention.

For the purposes of the present invention, “bioavailability” of a drug is defined as both the relative amount of drug from an administered dosage form which enters the systemic circulation and the rate at which the drug appears in the blood stream. Bioavailability is largely reflected by AUC, which is governed by at least 3 factors: (i) absorption which controls bioavailability, followed by (ii) its tissue re-distribution and (iii) elimination (metabolic degradation plus renal and other mechanisms).

“AUC” refers to the mean area under the plasma concentration-time curve; “AUC₀₋ₜ” refers to area under the concentration-time curve from time zero to the time of the last sample collection; “AUC₀₋₂₄” refers to area under the concentration-time curve from time zero to 24 hours; “AUC₀₋₄₈” refers to area under the concentration-time curve from time zero to 48 hours; “Cₘₐₓ” refers to maximum observed plasma concentration; “Tₘₐₓ” (or “tₘₐₓ”) refers to the time to achieve the Cₘₐₓ; “t₁2” refers to the apparent half-life and is calculated as (ln 2/Kₑₗ), where Kₑₗ refers to the apparent first-order elimination rate constant “absolute bioavailability” is the extent or fraction of drug absorbed upon extravascular administration in comparison to the dose size administered.

“Absolute bioavailability” is estimated by taking into consideration tissue re-distribution and biotransformation (i.e., elimination) which can be estimated in turn via intravenous administration of the drug. Unless otherwise indicated, “mean plasma concentration” and “plasma concentration” are used herein interchangeably; “HPLC” refers to high performance liquid chromatography; “pharmaceutically acceptable” refers to physiologically tolerable materials, which do not typically produce an allergic or other untoward reaction, such as gastric upset, dizziness and the like, when administered to a mammal; “mammal” refers to a class of higher vertebrates comprising man and all other animals that nourish their young with milk secreted by mammary glands and have the skin usually more or less covered with hair; and “treating” is intended to encompass relieving, alleviating or eliminating at least one symptom of a disease(s) in a mammal.

The term “treatment”, as used herein, is intended to encompass administration of compounds according to the invention prophylactically to prevent or suppress an undesired condition, and therapeutically to eliminate or reduce the extent or symptoms of the condition. Treatment according to the invention is given to a human or other mammal having a disease or condition creating a need of such treatment. Treatment also includes application of the compound to cells or organs in vitro. Treatment may be by systemic or local administration.

The catanionic vesicles of the present invention may be formulated into “pharmaceutical compositions” with appropriate pharmaceutically acceptable carriers, excipients or diluents. If appropriate, pharmaceutical compositions may be formulated into preparations including, but not limited to, solid, semi-solid, liquid, or gaseous forms, such as tablets, capsules, powders, granules, ointments, solutions, suppositories, injections, inhalants, and aerosols, in the usual ways for their respective route of administration.

An effective amount is the amount of active ingredient administered in a single dose or multiple doses necessary to achieve the desired pharmacological effect. A skilled practitioner can determine and optimize an effective dose for an individual patient or to treat an individual condition by routine experimentation and titration well known to the skilled clinician. The actual dose and schedule may vary depending on whether the compositions are administered in combination with other drugs, or depending on inter-individual differences in pharmacokinetics, drug disposition, and metabolism. Similarly, amounts may vary for in vitro applications. It is within the skill in the art to adjust the dose in accordance with the necessities of a particular situation without undue experimentation. Where disclosed herein, dose ranges do not preclude use of a higher or lower dose of a component, as might be warranted in a particular application.

The invention also provides for pharmaceutical compositions comprising as active material a catanionic vesicle bearing a bioconjugate, and optionally carrying a therapeutic agent according to the present invention together with one or more pharmaceutically acceptable carriers, excipients or diluents. Any conventional technique may be used for the preparation of pharmaceutical formulations according to the invention. The active ingredient may be contained in a formulation that provides quick release, sustained release or delayed release after administration to the patient. Pharmaceutical compositions that are useful in the methods of the invention may be prepared, packaged, or sold in formulations suitable for oral, parenteral and topical administration.

The formulations of the pharmaceutical compositions described herein may be prepared by any method known or hereafter developed. In general, preparation includes bringing the active ingredient into association with a carrier or one or more other additional components, and then, if necessary or desirable, shaping or packaging the product into a desired single- or multi-dose unit.

Prolonged activity is a valuable attribute of drugs in general and of anticonvulsant drugs in particular. Aside from allowing infrequent administration, it also improves patients' compliance with the drug. Furthermore, serum and tissue levels, which are crucial for maintaining therapeutic effectiveness, are more stable with a long acting compound. Moreover, stable serum levels reduce the incidence of side effects and/or other adverse effects.

As used herein, “additional components” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; pharmaceutically acceptable polymeric or hydrophobic materials as well as other components.

The descriptions of pharmaceutical compositions provided herein include pharmaceutical compositions which are suitable for administration to humans. It will be understood by the skilled artisan, based on this disclosure, that such compositions are generally suitable for administration to any mammal or other animal. Preparation of compositions suitable for administration to various animals is well understood, and the ordinarily skilled veterinary pharmacologist can design and perform such modifications with routine experimentation based on pharmaceutical compositions for administration to humans.

Furthermore, the compositions described herein, for example, bioconjugate bearing vesicles, can also be used for agricultural applications such as pesticide and fungicide application, and for other treatment of plants.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient in each unit dose is generally equal to the total amount of the active ingredient which would be administered or a convenient fraction of a total dosage amount such as, for example, one-half or one-third of such a dosage.

A formulation of a pharmaceutical composition of the invention suitable for oral administration may be in the form of a discrete solid dosage unit. Solid dosage units include, for example, a tablet, a caplet, a hard or soft capsule, a cachet, a troche, or a lozenge. Each solid dosage unit contains a predetermined amount of the active ingredient, for example a unit dose or fraction thereof. Other formulations suitable for administration include, but are not limited to, a powdered or granular formulation, an aqueous or oily suspension, an aqueous or oily solution, an emulsion, an aqueous liquor or a non-aqueous liquid may be employed, such as a syrup, an elixir, an emulsion, or a draught. As used herein, an “oily” liquid is one which comprises a carbon or silicon based liquid that is less polar than water. In such pharmaceutical dosage forms, the active agent preferably is utilized together with one or more pharmaceutically acceptable carrier(s) therefore and optionally any other therapeutic ingredients. The carrier(s) must be pharmaceutically acceptable in the sense of being compatible with the other ingredients of the formulation and not unduly deleterious to the recipient thereof

A tablet comprising the active ingredient may be made, for example, by compressing or molding the active ingredient, optionally containing one or more additional components. Compressed tablets may be prepared by compressing, in a suitable device, the so active ingredient in a free-flowing form such as a powder or granular preparation, optionally mixed with one or more of a binder, a lubricant, a glidant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Tablets may be non-coated or they may be coated using methods known in the art or methods to be developed. Coated tablets may be formulated for delayed disintegration in the gastrointestinal tract of a subject, for example, by use of an enteric coating, thereby providing sustained release and absorption of the active ingredient. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional components including, for example, an inert solid diluent. Soft gelatin capsules comprising the active ingredient may be made using a physiologically degradable composition, such as gelatin. Such soft capsules comprise the active ingredient, which may be mixed with water or an oil medium.

Liquid formulations of a pharmaceutical composition of the invention which are suitable for administration may be prepared, packaged, and sold either in liquid form or in the form of a dry product intended for reconstitution with water or another suitable vehicle prior to use.

Liquid suspensions, in which the active ingredient is dispersed in an aqueous or oily vehicle, and liquid solutions, in which the active ingredient is dissolved in an aqueous or oily vehicle, may be prepared using conventional methods or methods to be developed. Liquid suspension of the active ingredient may be in an aqueous or oily vehicle and may further include one or more additional components such as, for example, suspending agents, dispersing or wetting agents, emulsifying agents, demulcents, preservatives, buffers, salts, flavorings, coloring agents, and sweetening agents. Oily suspensions may further comprise a thickening agent. Liquid solutions of the active ingredient may be in an aqueous or oily vehicle and may further include one or more additional components such as, for example, preservatives, buffers, salts, flavorings, coloring agents, and sweetening agents.

To prepare such pharmaceutical dosage forms, one or more of the aforementioned compounds of formula (I) are intimately admixed with a pharmaceutical carrier according to conventional pharmaceutical compounding techniques. The carrier may take a wide variety of forms depending on the form of preparation desired for administration. In preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent a preferred oral dosage. If desired, tablets may be sugar coated or enteric coated by standard techniques.

The compositions of the present invention can be provided in unit dosage form, wherein each dosage unit, e.g., a teaspoon, tablet, capsule, solution, or suppository, contains a predetermined amount of the active drug or prodrug, alone or in appropriate combination with other pharmaceutically-active agents. The term “unit dosage form” refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically-acceptable diluent, carrier (e.g., liquid carrier such as a saline solution, a buffer solution, or other physiological aqueous solution), or vehicle, where appropriate.

Powdered and granular formulations according to the invention may be prepared using known methods or methods to be developed. Such formulations may be administered directly to a subject, or used, for example, to form tablets, to fill capsules, or to prepare an aqueous or oily suspension or solution by addition of an aqueous or oily vehicle thereto. Powdered or granular formulations may further comprise one or more of a dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. Such compositions may further comprise one or more emulsifying agents. These emulsions may also contain additional components including, for example, sweetening or flavoring agents.

A tablet may be made by compression or molding, or wet granulation, optionally with one or more accessory ingredients. Compressed tablets may be prepared by compressing the powder in a suitable machine, with the active compound being in a free-flowing form such as a powder or granules which optionally is mixed with a binder, disintegrant, lubricant, inert diluent, surface active agent, or discharging agent.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol. The formulations may be presented in unit-dose or multi-dose form.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

In addition to the aforementioned ingredients, formulations of this invention may further include one or more accessory ingredient(s) selected from diluents, buffers, flavoring agents, binders, disintegrants, surface active agents, thickeners, lubricants, preservatives (including antioxidants), and the like. The formulation of the present invention can have immediate release, sustained release, delayed-onset release or any other release profile known to one skilled in the art.

The invention also comprises an article of manufacture which is a container holding the pharmaceutical composition which comprises the catanionic vesicles bearing a bioconjugate associated with printed labeling instructions. The printed labeling can provide that the pharmaceutical composition should be administered either with food or within a defined period of time before or after ingestion of food. The composition will be contained in any suitable container capable of holding and dispensing the dosage form and which will not significantly interact with the composition. The labeling instructions will be consistent with the methods of treatment described herein. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A catanionic surfactant vesicle, comprising:
a bilayer comprising a cationic surfactant, an anionic surfactant, and a bioconjugate;
the bilayer having an inner surface and an outer surface;
the bioconjugate comprising a carbohydrate and/or peptide moiety and a hydrophobic group,
wherein at least a portion of the hydrophobic group is within the bilayer and
wherein the carbohydrate and/or peptide moiety is on the outer surface of the bilayer.

2. The catanionic surfactant vesicle of claim 1, wherein the bioconjugate is selected from the group consisting of a glycoconjugate, a lipid oligosaccharide, and a lipid polysaccharide.

3. The catanionic surfactant vesicle of claim 1, wherein the hydrophobic group comprises an alkyl chain.

4. The catanionic surfactant vesicle of claim 1,
further comprising a solute ion having a charge and an inner pool bounded by the inner surface of the bilayer,
wherein the solute ion having a charge is within the inner pool and/or the bilayer,
wherein the bilayer has a net surface charge, and
wherein the net surface charge of the bilayer is opposite to that of the solute ion.

5. The catanionic surfactant vesicle of claim 1,
further comprising a solute molecule or solute ion and an inner pool bounded by the inner surface of the bilayer,
wherein the solute molecule or solute ion is within the inner pool and/or the bilayer,
wherein the solute molecule or solute ion is selected from the group consisting of a dye, a radionuclide, a pharmaceutical agent, a biotherapeutic agent, a chemotherapeutic agent, a radiotherapeutic agent, and combinations.

6. The catanionic surfactant vesicle of claim 1,
further comprising a solute molecule or solute ion and an inner pool bounded by the inner surface of the bilayer,
wherein the solute molecule or solute ion is within the inner pool and/or the bilayer,
wherein the solute molecule or solute ion is selected from the group consisting of a metal, a natural product, a peptide, an oligopeptide, a polypeptide, a saccharide, an oligosaccharide, a polysaccharide, a nucleotide, an oligonucleotide, a polynucleotide, DNA, RNA, derivatives of these, and combinations.

7. The catanionic surfactant vesicle of claim 1,
further comprising a solute molecule or solute ion and an inner pool bounded by the inner surface of the bilayer,
wherein the solute molecule or solute ion is within the inner pool and/or the bilayer,
wherein the solute molecule or solute ion is selected from the group consisting of carboxyfluoroscein (CF), sulfarhodamine 101 (SR 101), Lucifer yellow (LY), rhodamine 6G (R6G) Doxorubicin, derivatives of these, and combinations.

8. The catanionic surfactant vesicle of claim 1,
further comprising a cell having a surface with a receptor,
wherein the carbohydrate and/or peptide moiety of the bioconjugate is bound to the receptor on the surface of the cell.

9. The catanionic surfactant vesicle of claim 1,
further comprising a lectin,
wherein the bioconjugate is a glycoconjugate,
wherein the carbohydrate moiety of the glycoconjugate is bound to the lectin.

10. A catanionic vesicle library, comprising:
at least two catanionic surfactant vesicles according to claim 1,
wherein each catanionic surfactant vesicle comprises an independently selected bioconjugate.

11. The catanionic vesicle library of claim 10,
wherein each catanionic surfactant vesicle further comprises an independently selected solute molecule or solute ion and an inner pool bounded by the inner surface of the bilayer,
wherein the solute molecule or solute ion is within the inner pool and/or the bilayer,
wherein the solute molecule or solute ion is selected from the group consisting of a dye, a radionuclide, a pharmaceutical agent, a chemotherapeutic agent, a radiotherapeutic agent, and combinations.

12. A lectin detection system,
comprising a catanionic surfactant vesicle according to claim 1,
wherein the bioconjugate is a glycoconjugate,
wherein the glycoconjugate of the catanionic surfactant vesicle binds to a predetermined lectin, and
wherein the first catanionic surfactant vesicle further comprises a dye.

13. A kit, comprising:
a premeasured amount of an anionic surfactant in a first labeled container;
a premeasured amount of a cationic surfactant in a second labeled container; and
a premeasured amount of a bioconjugate in a third labeled container,
wherein the premeasured amount of the anionic surfactant, the premeasured amount of the cationic surfactant, and the premeasured amount of the bioconjugate are selected so that when the premeasured amount of the anionic surfactant, the premeasured amount of the cationic surfactant, and the premeasured amount of the bioconjugate are added to a predetermined amount of water, catanionic surfactant vesicles are formed and
wherein the catanionic surfactant vesicles comprise a bilayer comprising the cationic surfactant, the anionic surfactant, and the bioconjugate.

14. A method of making a bioconjugate-decorated catanionic vesicle comprising:
providing an anionic surfactant, a cationic surfactant, and a bioconjugate comprising a carbohydrate and/or peptide moiety and a hydrophobic group; and
combining the anionic surfactant, the cationic surfactant, and the bioconjugate with water to form a bioconjugate-decorated catanionic vesicle having a bilayer with an inner surface and an outer surface that comprises the anionic surfactant and the cationic surfactant with at least a portion of the hydrophobic group within the bilayer and with the carbohydrate and/or peptide moiety on the outer surface of the bilayer.

15. The method of claim 14, further comprising:
providing a solute ion having a charge; and
combining the solute ion with the anionic surfactant, the cationic surfactant, the bioconjugate, and the water, so that the bilayer has a net surface charge, the catanionic vesicle comprises an inner pool bounded by the inner surface of the bilayer, the net surface charge of the bilayer is opposite to that of the solute ion, and the solute ion is within the inner pool and/or the bilayer.

16. A method for sequestering a solute ion within a bioconjugate-decorated catanionic vesicle, comprising:
determining the charge of the solute ion;
creating a bioconjugate-decorated catanionic vesicle having a net surface charge opposite to the charge of the solute ion according to the method of claim 18;
combining the catanionic vesicle with the solute ion; and
allowing the catanionic vesicle to sequester the solute ion,
wherein the bilayer has a net surface charge.

17. A method of introducing an agent into a cell, comprising:
contacting the cell with a composition comprising a catanionic surfactant vesicle comprising a bilayer of a cationic surfactant, an anionic surfactant, and a bioconjugate defining an inner pool, wherein the agent is sequestered in the bilayer and/or the inner pool,
wherein the cell comprises a lectin, a carbohydrate-binding, and/or a peptide-binding site that binds the bioconjugate.

18. The method of claim 17, wherein the agent is selected from the group consisting of a dye, a radionuclide, a pharmaceutical agent, a biotherapeutic agent, a chemotherapeutic agent, a radiotherapeutic agent, a metal, a natural product, a peptide, an oligopeptide, a polypeptide, a saccharide, an oligosaccharide, a polysaccharide, a nucleotide, an oligonucleotide, a polynucleotide, DNA, RNA, derivatives of these, and combinations.

19. A method of gene therapy, comprising introducing an agent into a cell according to the method of claim 17, wherein the agent is a nucleic acid.

20. A method for determining the separation distance of carbohydrate binding sites on a sample lectin, comprising:
providing a set of catanionic surfactant vesicles conjugated with a glycoconjugate comprising a carbohydrate moiety that is a ligand for the sample lectin over a range of glycoconjugate mole fractions;
determining the initial rate of reaction between each catanionic surfactant vesicle functionalized with the glycoconjugate in the set and the sample lectin by using a turbidity assay;
determining the value of carbohydrate binding site separation in a collision model that provides the best fit to the initial rate of reaction as a function of the mole fraction of glycoconjugate data;
taking the value of carbohydrate binding site separation in the collision model as representative of the separation distance of carbohydrate binding sites on the sample lectin.

21. A method of detecting receptors on a sample, comprising:
administering to the sample catanionic surfactant vesicles,
flushing away excess catanionic surfactant vesicles from the sample,
imaging a characteristic signal of a label of the catanionic surfactant vesicles,
associating regions displaying the characteristic signal of the label with binding of the catanionic surfactant vesicles and presence of the receptors on the sample,
wherein the catanionic surfactant vesicles comprise a bilayer having an inner surface and an outer surface comprising a cationic surfactant, an anionic surfactant, and a bioconjugate, the bioconjugate comprising a carbohydrate and/or peptide moiety and a hydrophobic group, at least a portion of the hydrophobic group within the bilayer and the carbohydrate and/or peptide moiety on the outer surface,
wherein the inner surface bounds an inner pool,
wherein the label is sequestered in the bilayer and/or the inner pool, and
wherein the carbohydrate and/or peptide moiety is capable of binding with the receptor of the sample.

22. A method of detecting cancer cells in a subject, comprising:
administering to the subject catanionic surfactant vesicles in a physiologically acceptable carrier;
allowing the catanionic surfactant vesicles to bind with receptors on the cancer cells;
imaging a characteristic signal of a label of the catanionic surfactant vesicles,
associating regions of the subject displaying the characteristic signal of the label with binding of the catanionic surfactant vesicles and the presence of cancer cells,
wherein the catanionic surfactant vesicles comprise a bilayer having an inner surface and an outer surface comprising a cationic surfactant, an anionic surfactant, and a bioconjugate, the bioconjugate comprising a carbohydrate and/or peptide moiety and a hydrophobic group, at least a portion of the hydrophobic group within the bilayer and the carbohydrate and/or peptide moiety on the outer surface,
wherein the inner surface bounds an inner pool,
wherein the label is sequestered in the bilayer and/or the inner pool, and
wherein the carbohydrate and/or peptide moiety is capable of binding with the receptors on the cancer cells.

23. A method of treating cancer in a subject, comprising:
administering to the subject catanionic surfactant vesicles in a physiologically acceptable carrier; and
allowing the catanionic surfactant vesicles to bind with receptors on the cancer cells;
wherein the catanionic surfactant vesicles comprise a bilayer having an inner surface and an outer surface comprising a cationic surfactant, an anionic surfactant, and a bioconjugate, the bioconjugate comprising a carbohydrate and/or peptide moiety and a hydrophobic group, at least a portion of the hydrophobic group within the bilayer and the carbohydrate and/or peptide moiety on the outer surface,
wherein the inner surface bounds an inner pool,
wherein a chemotherapeutic, radiotherapeutic, and/or biotherapeutic agent is sequestered in the bilayer and/or the inner pool, and
wherein the carbohydrate and/or peptide moiety is capable of binding with the receptors on the cancer cells.
